SEQUENCE LISTING (133 sequences)

SEQ ID NO: 1 - DNA, 12 bp, artificial sequence
  synthesized oligos with DNA & RNA, connected by phosphothiorate bonds
uactcggtctcg

SEQ ID NO: 2 - DNA, 12 bp, artificial sequence
  synthesized oligos with DNA & RNA, connected by phosphothiorate bonds
uactcagtctcg

SEQ ID NO: 3 - DNA, 12 bp, artificial sequence
  synthesized oligos with DNA & RNA, connected by phosphothiorate bonds
agaaacaaatuc

SEQ ID NO: 4 - DNA, 12 bp, artificial sequence
  synthesized oligos with DNA & RNA, connected by phosphothiorate bonds
agaaagaaatuc

SEQ ID NO: 5 - DNA, 12 bp, artificial sequence
  synthesized oligos with DNA & RNA, connected by phosphothiorate bonds
agagacttctuu

SEQ ID NO: 6 - DNA, 12 bp, artificial sequence
  synthesized oligos with DNA & RNA, connected by phosphothiorate bonds
agagagttctuu

SEQ ID NO: 7 - DNA, 12 bp, artificial sequence
  synthesized oligos with DNA & RNA, connected by phosphothiorate bonds
ucagcgtcatca

SEQ ID NO: 8 - DNA, 12 bp, artificial sequence
  synthesized oligos with DNA & RNA, connected by phosphothiorate bonds
ucagcatcatca

SEQ ID NO: 9 - DNA, 12 bp, artificial sequence
  synthesized oligos with DNA & RNA, connected by phosphothiorate bonds
aagaccagccug

SEQ ID NO: 10 - DNA, 12 bp, artificial sequence
  synthesized oligos with DNA & RNA, connected by phosphothiorate bonds
aagactagccug

SEQ ID NO: 11 - DNA, 12 bp, artificial sequence
  synthesized oligos with DNA & RNA, connected by phosphothiorate bonds
ccagggactccu

SEQ ID NO: 12 - DNA, 12 bp, artificial sequence
  synthesized oligos with DNA & RNA, connected by phosphothiorate bonds
ccaggcactccu

SEQ ID NO: 13 - DNA, 12 bp, artificial sequence
  synthesized oligos with DNA & RNA, connected by phosphothiorate bonds
gccttcccctgc

SEQ ID NO: 14 - DNA, 12 bp, artificial sequence
  synthesized oligos with DNA & RNA, connected by phosphothiorate bonds
gcctttccctgc

SEQ ID NO: 15 - DNA, 12 bp, artificial sequence
  synthesized oligos with DNA & RNA, connected by phosphothiorate bonds
uggatagtctuu

SEQ ID NO: 16 - DNA, 12 bp, artificial sequence
  synthesized oligos with DNA & RNA, connected by phosphothiorate bonds
uggatggtctuu

SEQ ID NO: 17 - DNA, 12 bp, artificial sequence
  synthesized oligos with DNA & RNA, connected by phosphothiorate bonds
ggugtaggaggu

SEQ ID NO: 18 - DNA, 12 bp, artificial sequence
  synthesized oligos with DNA & RNA, connected by phosphothiorate bonds
ggugtgggaggu

SEQ ID NO: 19 - DNA, 12 bp, artificial sequence
  synthesized oligos with DNA & RNA, connected by phosphothiorate bonds
uuggcaatgauc

SEQ ID NO: 20 - DNA, 12 bp, artificial sequence
  synthesized oligos with DNA & RNA, connected by phosphothiorate bonds
uuggcgatgauc

SEQ ID NO: 21 - DNA, 12 bp, artificial sequence
  synthesized oligos with DNA & RNA, connected by phosphothiorate bonds
ugctctgccagc

SEQ ID NO: 22 - DNA, 12 bp, artificial sequence
  synthesized oligos with DNA & RNA, connected by phosphothiorate bonds
ugctccgccagc

SEQ ID NO: 23 - DNA, 12 bp, artificial sequence
  synthesized oligos with DNA & RNA, connected by phosphothiorate bonds
gugccttctggc

SEQ ID NO: 24 - DNA, 12 bp, artificial sequence
  synthesized oligos with DNA & RNA, connected by phosphothiorate bonds
gugccgtctggc

SEQ ID NO: 25 - DNA, 12 bp, artificial sequence
  synthesized oligos with DNA & RNA, connected by phosphothiorate bonds
gugccatctggc

SEQ ID NO: 26 - DNA, 12 bp, artificial sequence
  synthesized oligos with DNA & RNA, connected by phosphothiorate bonds
uaggtgagctug

SEQ ID NO: 27 - DNA, 12 bp, artificial sequence
  synthesized oligos with DNA & RNA, connected by phosphothiorate bonds
uaggtaagctug

SEQ ID NO: 28 - DNA, 12 bp, artificial sequence
  synthesized oligos with DNA & RNA, connected by phosphothiorate bonds
gcacatggttgu

SEQ ID NO: 29 - DNA, 12 bp, artificial sequence
  synthesized oligos with DNA & RNA, connected by phosphothiorate bonds
gcacacggttgu

SEQ ID NO: 30 - DNA, 12 bp, artificial sequence
  synthesized oligos with DNA & RNA, connected by phosphothiorate bonds
caucccggggcu

SEQ ID NO: 31 - DNA, 12 bp, artificial sequence
  synthesized oligos with DNA & RNA, connected by phosphothiorate bonds
cauccaggggcu

SEQ ID NO: 32 - DNA, 12 bp, artificial sequence
  synthesized oligos with DNA & RNA, connected by phosphothiorate bonds
aggcagggcgac

SEQ ID NO: 33 - DNA, 12 bp, artificial sequence
  synthesized oligos with DNA & RNA, connected by phosphothiorate bonds
aggcaggggcga

SEQ ID NO: 34 - DNA, 12 bp, artificial sequence
  synthesized oligos with DNA & RNA, connected by phosphothiorate bonds
uccttcctctuc

SEQ ID NO: 35 - DNA, 12 bp, artificial sequence
  synthesized oligos with DNA & RNA, connected by phosphothiorate bonds
uccttgctctuc

SEQ ID NO: 36 - DNA, 12 bp, artificial sequence
  synthesized oligos with DNA & RNA, connected by phosphothiorate bonds
caucagttccca

SEQ ID NO: 37 - DNA, 12 bp, artificial sequence
  synthesized oligos with DNA & RNA, connected by phosphothiorate bonds
caucacttccca

SEQ ID NO: 38 - DNA, 12 bp, artificial sequence
  synthesized oligos with DNA & RNA, connected by phosphothiorate bonds
cgcccgcacccu

SEQ ID NO: 39 - DNA, 12 bp, artificial sequence
  synthesized oligos with DNA & RNA, connected by phosphothiorate bonds
cgcccacacccu

SEQ ID NO: 40 - DNA, 12 bp, artificial sequence
  synthesized oligos with DNA & RNA, connected by phosphothiorate bonds
ucccatggggua

SEQ ID NO: 41 - DNA, 12 bp, artificial sequence
  synthesized oligos with DNA & RNA, connected by phosphothiorate bonds
ucccacggggua

SEQ ID NO: 42 - DNA, 12 bp, artificial sequence
  synthesized oligos with DNA & RNA, connected by phosphothiorate bonds
uuugtgcctguu

SEQ ID NO: 43 - DNA, 12 bp, artificial sequence
  synthesized oligos with DNA & RNA, connected by phosphothiorate bonds
uuugtccctguu

SEQ ID NO: 44 - DNA, 12 bp, artificial sequence
  synthesized oligos with DNA & RNA, connected by phosphothiorate bonds
ccagttgggtca

SEQ ID NO: 45 - DNA, 12 bp, artificial sequence
  synthesized oligos with DNA & RNA, connected by phosphothiorate bonds
ccagtcgggtca

SEQ ID NO: 46 - DNA, 12 bp, artificial sequence
  synthesized oligos with DNA & RNA, connected by phosphothiorate bonds
guuaaccgggcu

SEQ ID NO: 47 - DNA, 12 bp, artificial sequence
  synthesized oligos with DNA & RNA, connected by phosphothiorate bonds
guuaatcgggcu

SEQ ID NO: 48 - DNA, 12 bp, artificial sequence
  synthesized oligos with DNA & RNA, connected by phosphothiorate bonds
ucutatttgtgu

SEQ ID NO: 49 - DNA, 12 bp, artificial sequence
  synthesized oligos with DNA & RNA, connected by phosphothiorate bonds
ucutattttgug

SEQ ID NO: 50 - DNA, 12 bp, artificial sequence
  synthesized oligos with DNA & RNA, connected by phosphothiorate bonds
ccaccgtgccug

SEQ ID NO: 51 - DNA, 12 bp, artificial sequence
  synthesized oligos with DNA & RNA, connected by phosphothiorate bonds
ccaccatgccug

SEQ ID NO: 52 - DNA, 12 bp, artificial sequence
  synthesized oligos with DNA & RNA, connected by phosphothiorate bonds
gcctacctttgu

SEQ ID NO: 53 - DNA, 12 bp, artificial sequence
  synthesized oligos with DNA & RNA, connected by phosphothiorate bonds
gcctaactttgu

SEQ ID NO: 54 - DNA, 12 bp, artificial sequence
  synthesized oligos with DNA & RNA, connected by phosphothiorate bonds
uccccacagacc

SEQ ID NO: 55 - DNA, 12 bp, artificial sequence
  synthesized oligos with DNA & RNA, connected by phosphothiorate bonds
ucccccacagac

SEQ ID NO: 56 - DNA, 12 bp, artificial sequence
  synthesized oligos with DNA & RNA, connected by phosphothiorate bonds
ccattatgacac

SEQ ID NO: 57 - DNA, 12 bp, artificial sequence
  synthesized oligos with DNA & RNA, connected by phosphothiorate bonds
ccattgtgacac

SEQ ID NO: 58 - DNA, 12 bp, artificial sequence
  synthesized oligos with DNA & RNA, connected by phosphothiorate bonds
cuactcaggagg

SEQ ID NO: 59 - DNA, 12 bp, artificial sequence
  synthesized oligos with DNA & RNA, connected by phosphothiorate bonds
cuacttaggagg

SEQ ID NO: 60 - DNA, 12 bp, artificial sequence
  synthesized oligos with DNA & RNA, connected by phosphothiorate bonds
ccaggcgtggug

SEQ ID NO: 61 - DNA, 12 bp, artificial sequence
  synthesized oligos with DNA & RNA, connected by phosphothiorate bonds
ccaggggtggug

SEQ ID NO: 62 - DNA, 12 bp, artificial sequence
  synthesized oligos with DNA & RNA, connected by phosphothiorate bonds
acugcacccagc

SEQ ID NO: 63 - DNA, 12 bp, artificial sequence
  synthesized oligos with DNA & RNA, connected by phosphothiorate bonds
acugcgcccagc

SEQ ID NO: 64 - DNA, 12 bp, artificial sequence
  synthesized oligos with DNA & RNA, connected by phosphothiorate bonds
aaacaaaccaaa

SEQ ID NO: 65 - DNA, 12 bp, artificial sequence
  synthesized oligos with DNA & RNA, connected by phosphothiorate bonds
aaacagaccaaa

SEQ ID NO: 66 - DNA, 12 bp, artificial sequence
  synthesized oligos with DNA & RNA, connected by phosphothiorate bonds
cagccgggccaa

SEQ ID NO: 67 - DNA, 12 bp, artificial sequence
  synthesized oligos with DNA & RNA, connected by phosphothiorate bonds
cagcctggccaa

SEQ ID NO: 68 - DNA, 12 bp, artificial sequence
  synthesized oligos with DNA & RNA, connected by phosphothiorate bonds
aacaaaccaaau

SEQ ID NO: 69 - DNA, 12 bp, artificial sequence
  synthesized oligos with DNA & RNA, connected by phosphothiorate bonds
aacaaacagacc

SEQ ID NO: 70 - DNA, 12 bp, artificial sequence
  synthesized oligos with DNA & RNA, connected by phosphothiorate bonds
gggggcaagcag

SEQ ID NO: 71 - DNA, 12 bp, artificial sequence
  synthesized oligos with DNA & RNA, connected by phosphothiorate bonds
gggggagcaagc

SEQ ID NO: 72 - DNA, 12 bp, artificial sequence
  synthesized oligos with DNA & RNA, connected by phosphothiorate bonds
ccatcgcaaaaa

SEQ ID NO: 73 - DNA, 12 bp, artificial sequence
  synthesized oligos with DNA & RNA, connected by phosphothiorate bonds
ccatctcaaaaa

SEQ ID NO: 74 - DNA, 12 bp, artificial sequence
  synthesized oligos with DNA & RNA, connected by phosphothiorate bonds
aacccacttccu

SEQ ID NO: 75 - DNA, 12 bp, artificial sequence
  synthesized oligos with DNA & RNA, connected by phosphothiorate bonds
aacccgcttccu

SEQ ID NO: 76 - DNA, 12 bp, artificial sequence
  synthesized oligos with DNA & RNA, connected by phosphothiorate bonds
cacctatagtcc

SEQ ID NO: 77 - DNA, 12 bp, artificial sequence
  synthesized oligos with DNA & RNA, connected by phosphothiorate bonds
cacctgtagtcc

SEQ ID NO: 78 - DNA, 12 bp, artificial sequence
  synthesized oligos with DNA & RNA, connected by phosphothiorate bonds
gugtcttcctuc

SEQ ID NO: 79 - DNA, 12 bp, artificial sequence
  synthesized oligos with DNA & RNA, connected by phosphothiorate bonds
gugtcctcctuc

SEQ ID NO: 80 - DNA, 12 bp, artificial sequence
  synthesized oligos with DNA & RNA, connected by phosphothiorate bonds
cugagggaggag

SEQ ID NO: 81 - DNA, 12 bp, artificial sequence
  synthesized oligos with DNA & RNA, connected by phosphothiorate bonds
cugagtgaggag

SEQ ID NO: 82 - DNA, 12 bp, artificial sequence
  synthesized oligos with DNA & RNA, connected by phosphothiorate bonds
ccgcgccgctcc

SEQ ID NO: 83 - DNA, 12 bp, artificial sequence
  synthesized oligos with DNA & RNA, connected by phosphothiorate bonds
ccgcgacgctcc

SEQ ID NO: 84 - DNA, 12 bp, artificial sequence
  synthesized oligos with DNA & RNA, connected by phosphothiorate bonds
uaucagaggtau

SEQ ID NO: 85 - DNA, 12 bp, artificial sequence
  synthesized oligos with DNA & RNA, connected by phosphothiorate bonds
uaucataggtau

SEQ ID NO: 86 - DNA, 12 bp, artificial sequence
  synthesized oligos with DNA & RNA, connected by phosphothiorate bonds
ccutagcagtcu

SEQ ID NO: 87 - DNA, 12 bp, artificial sequence
  synthesized oligos with DNA & RNA, connected by phosphothiorate bonds
ccutaccagtcu

SEQ ID NO: 88 - DNA, 12 bp, artificial sequence
  synthesized oligos with DNA & RNA, connected by phosphothiorate bonds
agatggagcaag

SEQ ID NO: 89 - DNA, 12 bp, artificial sequence
  synthesized oligos with DNA & RNA, connected by phosphothiorate bonds
agatgtagcaag

SEQ ID NO: 90 - DNA, 12 bp, artificial sequence
  synthesized oligos with DNA & RNA, connected by phosphothiorate bonds
ccuccacgtagu

SEQ ID NO: 91 - DNA, 12 bp, artificial sequence
  synthesized oligos with DNA & RNA, connected by phosphothiorate bonds
ccuccgcgtagu

SEQ ID NO: 92 - DNA, 12 bp, artificial sequence
  synthesized oligos with DNA & RNA, connected by phosphothiorate bonds
uactttgtctuu

SEQ ID NO: 93 - DNA, 12 bp, artificial sequence
  synthesized oligos with DNA & RNA, connected by phosphothiorate bonds
uactttctgtcu

SEQ ID NO: 94 - DNA, 12 bp, artificial sequence
  synthesized oligos with DNA & RNA, connected by phosphothiorate bonds
cuuaagtataau

SEQ ID NO: 95 - DNA, 12 bp, artificial sequence
  synthesized oligos with DNA & RNA, connected by phosphothiorate bonds
cuuaaatataau

SEQ ID NO: 96 - DNA, 12 bp, artificial sequence
  synthesized oligos with DNA & RNA, connected by phosphothiorate bonds
ccaactgcaaga

SEQ ID NO: 97 - DNA, 12 bp, artificial sequence
  synthesized oligos with DNA & RNA, connected by phosphothiorate bonds
ccaaccgcaaga

SEQ ID NO: 98 - DNA, 12 bp, artificial sequence
  synthesized oligos with DNA & RNA, connected by phosphothiorate bonds
agutacagccac

SEQ ID NO: 99 - DNA, 12 bp, artificial sequence
  synthesized oligos with DNA & RNA, connected by phosphothiorate bonds
agutaacagcca

SEQ ID NO: 100 - DNA, 12 bp, artificial sequence
  synthesized oligos with DNA & RNA, connected by phosphothiorate bonds
agacagggaggg

SEQ ID NO: 101 - DNA, 12 bp, artificial sequence
  synthesized oligos with DNA & RNA, connected by phosphothiorate bonds
agacacggaggg

SEQ ID NO: 102 - DNA, 12 bp, artificial sequence
  synthesized oligos with DNA & RNA, connected by phosphothiorate bonds
gautttcactga

SEQ ID NO: 103 - DNA, 12 bp, artificial sequence
  synthesized oligos with DNA & RNA, connected by phosphothiorate bonds
gauttccactga

SEQ ID NO: 104 - DNA, 12 bp, artificial sequence
  synthesized oligos with DNA & RNA, connected by phosphothiorate bonds
ggagagcagaca

SEQ ID NO: 105 - DNA, 12 bp, artificial sequence
  synthesized oligos with DNA & RNA, connected by phosphothiorate bonds
ggagaacagaca

SEQ ID NO: 106 - DNA, 12 bp, artificial sequence
  synthesized oligos with DNA & RNA, connected by phosphothiorate bonds
cuatatagaaau

SEQ ID NO: 107 - DNA, 12 bp, artificial sequence
  synthesized oligos with DNA & RNA, connected by phosphothiorate bonds
cuatacagaaau

SEQ ID NO: 108 - DNA, 12 bp, artificial sequence
  synthesized oligos with DNA & RNA, connected by phosphothiorate bonds
aggacggtgcuc

SEQ ID NO: 109 - DNA, 12 bp, artificial sequence
  synthesized oligos with DNA & RNA, connected by phosphothiorate bonds
aggacagtgcuc

SEQ ID NO: 110 - DNA, 12 bp, artificial sequence
  synthesized oligos with DNA & RNA, connected by phosphothiorate bonds
uuuatttgacac

SEQ ID NO: 111 - DNA, 12 bp, artificial sequence
  synthesized oligos with DNA & RNA, connected by phosphothiorate bonds
uuuatctgacac

SEQ ID NO: 112 - DNA, 12 bp, artificial sequence
  synthesized oligos with DNA & RNA, connected by phosphothiorate bonds
ugcccgggagcc

SEQ ID NO: 113 - DNA, 12 bp, artificial sequence
  synthesized oligos with DNA & RNA, connected by phosphothiorate bonds
ugcccaggagcc

SEQ ID NO: 114 - DNA, 12 bp, artificial sequence
  synthesized oligos with DNA & RNA, connected by phosphothiorate bonds
gcactcgggaca

SEQ ID NO: 115 - DNA, 12 bp, artificial sequence
  synthesized oligos with DNA & RNA, connected by phosphothiorate bonds
gcacttgggaca

SEQ ID NO: 116 - DNA, 12 bp, artificial sequence
  synthesized oligos with DNA & RNA, connected by phosphothiorate bonds
agcagaagagcc

SEQ ID NO: 117 - DNA, 12 bp, artificial sequence
  synthesized oligos with DNA & RNA, connected by phosphothiorate bonds
agcaggagagcc

SEQ ID NO: 118 - DNA, 12 bp, artificial sequence
  synthesized oligos with DNA & RNA, connected by phosphothiorate bonds
aggcaccgtgug

SEQ ID NO: 119 - DNA, 12 bp, artificial sequence
  synthesized oligos with DNA & RNA, connected by phosphothiorate bonds
aggcatcgtgug

SEQ ID NO: 120 - DNA, 12 bp, artificial sequence
  synthesized oligos with DNA & RNA, connected by phosphothiorate bonds
augaatgaccug

SEQ ID NO: 121 - DNA, 12 bp, artificial sequence
  synthesized oligos with DNA & RNA, connected by phosphothiorate bonds
augaacgaccug

SEQ ID NO: 122 - DNA, 12 bp, artificial sequence
  synthesized oligos with DNA & RNA, connected by phosphothiorate bonds
ccaccgcaccca

SEQ ID NO: 123 - DNA, 12 bp, artificial sequence
  synthesized oligos with DNA & RNA, connected by phosphothiorate bonds
ccaccacaccca

SEQ ID NO: 124 - DNA, 12 bp, artificial sequence
  synthesized oligos with DNA & RNA, connected by phosphothiorate bonds
aucccagaagcu

SEQ ID NO: 125 - DNA, 12 bp, artificial sequence
  synthesized oligos with DNA & RNA, connected by phosphothiorate bonds
aucccggaagcu

SEQ ID NO: 126 - DNA, 12 bp, artificial sequence
  synthesized oligos with DNA & RNA, connected by phosphothiorate bonds
augcccgactaa

SEQ ID NO: 127 - DNA, 12 bp, artificial sequence
  synthesized oligos with DNA & RNA, connected by phosphothiorate bonds
augcctgactaa

SEQ ID NO: 128 - DNA, 12 bp, artificial sequence
  synthesized oligos with DNA & RNA, connected by phosphothiorate bonds
gcaactctggag

SEQ ID NO: 129 - DNA, 12 bp, artificial sequence
  synthesized oligos with DNA & RNA, connected by phosphothiorate bonds
gcaacactggag

SEQ ID NO: 130 - DNA, 12 bp, artificial sequence
  synthesized oligos with DNA & RNA, connected by phosphothiorate bonds
ugagcgagggac

SEQ ID NO: 131 - DNA, 12 bp, artificial sequence
  synthesized oligos with DNA & RNA, connected by phosphothiorate bonds
ugagctagggac

SEQ ID NO: 132 - DNA, 12 bp, artificial sequence
  synthesized oligos with DNA & RNA, connected by phosphothiorate bonds
aagacacctgug

SEQ ID NO: 133 - DNA, 12 bp, artificial sequence
  synthesized oligos with DNA & RNA, connected by phosphothiorate bonds
aagacgcctgug

(19) United States
(12) Patent Application Publication
(10) Pub. No.: US 2016/0298114 A1
(43) Pub. Date: Oct. 13, 2016

(54) Haplotype Based Generalizable Allele Specific Silencing for Therapy of Cardiovascular Disease

(71) Applicant: The Board of Trustees of the Leland Stanford Junior University, Stanford, CA (US)

(72) Inventors: Matthew Wheeler, Sunnyvale, CA (US); Euan A. Ashley, Menlo Park, CA (US); Alexandra Dainis, Palo Alto, CA (US)

(73) Assignee: The Board of Trustees of the Leland Stanford Junior University, Stanford, CA (US)

(21) Appl. No.: 15/075,057

(22) Filed: Mar. 18, 2016

Related U.S. Application Data
(60) Provisional application No. 62/134,970, filed on Mar. 18, 2015.

(51) Int. Cl. C12N 15/113; G06F 19/18; G06F 19/22; C12Q 1/68

(52) U.S. Cl. CPC C12N 15/113; C12Q 1/6883; G06F 19/18; G06F 19/22; C12Q 2600/156; C12Q 2600/178; C12N 2310/14; C12N 2310/321; C12N 2320/34; A61K 48/00

(57) ABSTRACT

RNAi therapeutic systems in accordance with various embodiments of the invention provide for techniques for silencing expression of deleterious alleles using RNAi therapeutics targeting common variants of alleles. In many embodiments, processes and workflows for identifying common variants of alleles according to repeatedly occurring sets of SNPs are provided. The common variants can be found on genes where deleterious mutations can occur. The common variants can be the basis for targeting with RNAi therapeutics. Thereby, some embodiments of the invention enable efficient and cost saving targeting of common variants using RNAi therapeutics as opposed to individualized deleterious mutation targeting. Several embodiments of the invention further provide for processes for sequencing and phasing subject samples. After sequencing and phasing, some embodiments can apply the common variant targeted RNAi therapeutics to treat deleterious mutations.

### CROSS-REFERENCE TO RELATED APPLICATIONS

The present application claims priority under 35 U.S.C. §119(e) to U.S. Provisional Patent Application Ser. No. 62/134,970 entitled “Haplotype Based Generalizable Allele Specific Silencing for Therapy of Cardiovascular Disease” filed Mar. 18, 2015. The disclosure of U.S. Provisional Patent Application Ser. No. 62/134,970 is hereby incorporated by reference in its entirety.

### GOVERNMENT LICENSE RIGHTS

This invention was made with Government support under contracts DP2 OD004613 awarded by the National Institutes of Health and the Graduate Research Fellowships Program; and Fellow ID: 2014189226, awarded by the National Science Foundation. The Government has certain rights in this invention.

### FIELD OF THE INVENTION

The present invention generally relates to techniques for silencing expression of deleterious alleles using RNAi therapeutics targeting common variants of alleles.

### BACKGROUND

Hypertrophic cardiomyopathy (HCM) is a leading cause of early sudden cardiac death. There are no currently approved disease-modifying therapeutics to ameliorate cardiac structural changes or improve survival. Additionally, more than half of patients with familial cardiomyopathy do not have an identified genetic cause. Palliative therapies can mitigate risks of heart attacks via expensive and invasive surgical techniques. These techniques include septal application that causes planned heart attacks. Heart transplants are also an option for treatment. These options are expensive and involve significant surgeries. Recent improvements to RNAi therapeutics have opened up new avenues for treatment, but suffer from variable genetic links to HCM. Different patients often have different variant mutations which are associated with HCM. Individualized haplotype analysis suffers from being costly and time consuming. Next generation sequencing strategies have greatly improved the ability to identify patients at risk for developing inherited cardiomyopathies.

### SUMMARY OF THE INVENTION

Systems and methods for application identification in accordance with embodiments of the invention are disclosed. In one embodiment, method of downregulating expression of deleteriously mutated alleles using RNAi therapeutics targeting common variants of alleles using an RNAi therapeutics system is provided. The method receives subject samples using an RNAi therapeutics system. The subject samples include a set of alleles from the subject. The method sequences the subject samples using the RNAi therapeutics system. The method phases the sequenced samples using the RNAi therapeutics system. The method identifies a deleterious mutation on a particular allele from the set of alleles in the phased samples using the RNAi therapeutics system. The method identifies a common variant in phase with the deleterious mutation on the particular allele using the RNAi therapeutics system. The method selects an RNAi therapeutic targeting the common variant using the RNAi therapeutics system. The method applies the selected RNAi therapeutic utilizing a vector and the RNAi therapeutics system.

In a further embodiment, the method further includes selecting a disease using the RNAi therapeutics system, identifying a gene upon which mutations can cause the selected disease using the RNAi therapeutics system, identifying common variants of the gene using the RNAi therapeutics system, and preparing RNAi therapeutics that target the identified common variants using the RNAi therapeutics system.

In another embodiment, sequencing the subject samples further includes performing long read sequencing of the subject samples.

In a still further embodiment, the selected RNAi therapeutic includes a therapeutic composition that includes a suitable vector, and a nucleic acid compound with an antisense sequence complementary to RNA. The nucleic acid compound includes 2′-O-Methylated RNA bases and phosphorothioate Bonds and the antisense sequence is one of: UACTCGGTCTCG, UACTCAGTCTCG, AGAAACAAATUC, AGAAAGAAATUC, AGAGACTTCTUU, AGAGAGTTCTUU, UCAGCGTCATCA, UCAGCATCATCA, AAGACCAGCCUG, AAGACTAGCCUG, CCAGGGACTCCU, CCAGGCACTCCU, GCCTTCCCCTGC, GCCTTTCCCTGC, UGGATAGTCTUU, UGGATGGTCTUU, GGUGTAGGAGGU, GGUGTGGGAGGU, UUGGCAATGAUC, UUGGCGATGAUC, UGCTCTGCCAGC, UGCTCCGCCAGC, GUGCCTTCTGGC, GUGCCGTCTGGC, GUGCCATCTGGC, UAGGTGAGCTUG, and UAGGTAAGCTUG.

In still another embodiment, the antisense sequence complementary to RNA is an antisense oligonucleotide targeting common variants of the MYH7 gene.

In a yet further embodiment, the received subject samples are from a patient with hypertrophic cardiomyopathy that has a deleteriously mutated gene.

In yet another embodiment, the deleteriously mutated gene is MYH7 and the mutation is R403Q.

In a further embodiment again, the identified common variant is one of the following: rs2069540 reference genotype, rs2069540 alternate genotype, rs58290801 reference genotype, rs58290801 alternate genotype, rs2239577 reference genotype, rs2239577 alternate genotype, rs2231124 reference genotype, rs2231124 alternate genotype, rs7145023 reference genotype, rs7145023 alternate genotype, rs3729823 reference genotype, rs3729823 alternate genotype, rs201797477 reference genotype, rs201797477 alternate genotype, rs2754155 reference genotype, rs2754155 alternate genotype, rs1951154 reference genotype, rs1951154 alternate genotype, rs7157716 reference genotype, rs7157716 alternate genotype, rs3729830 reference genotype, rs3729830 alternate genotype, rs2231126 reference genotype, rs2231126 alternate genotype, rs2231126 reference genotype, rs144420313 alternate genotype, and rs144420313 reference genotype.

In another embodiment again, the vector comprises a transfection reagent.

In yet one another still yet embodiment, an RNAi therapeutic system is provided. The RNAi therapeutic system includes at least one processing unit and a memory storing a RNAi therapeutic application. The RNAi therapeutic application directs the at least one processing unit to receive subject samples, the subject samples including a set of alleles from the subject, sequence the subject samples, phase the sequenced samples, identify a deleterious mutation on a particular allele from the set of alleles in the phased samples, identify a common variant in phase with the deleterious mutation on the particular allele, select an RNAi therapeutic targeting the common variant, and apply the selected RNAi therapeutic utilizing a vector.

In a further additional embodiment, the RNAi therapeutic application further directs the at least one processing unit to select a disease, identify a gene upon which mutations can cause the selected disease, identify common variants of the gene, and prepare RNAi therapeutics that target the identified common variants.

In another additional embodiment, sequencing the subject samples further comprising performing long read sequencing of the subject samples.

In a still yet further embodiment, the selected RNAi therapeutic includes a therapeutic composition that includes a suitable vector, and a nucleic acid compound with an antisense sequence complementary to RNA. The nucleic acid compound includes 2′-O-Methylated RNA bases and phosphorothioate Bonds and the antisense sequence is one of: UACTCGGTCTCG, UACTCAGTCTCG, AGAAACAAATUC, AGAAAGAAATUC, AGAGACTTCTUU, AGAGAGTTCTUU, UCAGCGTCATCA, UCAGCATCATCA, AAGACCAGCCUG, AAGACTAGCCUG, CCAGGGACTCCU, CCAGGCACTCCU, GCCTTCCCCTGC, GCCTTTCCCTGC, UGGATAGTCTUU, UGGATGGTCTUU, GGUGTAGGAGGU, GGUGTGGGAGGU, UUGGCAATGAUC, UUGGCGATGAUC, UGCTCTGCCAGC, UGCTCCGCCAGC, GUGCCTTCTGGC, GUGCCGTCTGGC, GUGCCATCTGGC, UAGGTGAGCTUG, and UAGGTAAGCTUG.

In still yet another embodiment, the antisense sequence complementary to RNA is an antisense oligonucleotide targeting common variants of the MYH7 gene.

In a still further embodiment again, the received subject samples are from a patient with hypertrophic cardiomyopathy that has a deleteriously mutated gene.

In still another embodiment again, the deleteriously mutated gene is MYH7 and the mutation is R403Q.

In a still further additional embodiment, the identified common variant is one of the following: rs2069540 reference genotype, rs2069540 alternate genotype, rs58290801 reference genotype, rs58290801 alternate genotype, rs2239577 reference genotype, rs2239577 alternate genotype, rs2231124 reference genotype, rs2231124 alternate genotype, rs7145023 reference genotype, rs7145023 alternate genotype, rs3729823 reference genotype, rs3729823 alternate genotype, rs201797477 reference genotype, rs201797477 alternate genotype, rs2754155 reference genotype, rs2754155 alternate genotype, rs1951154 reference genotype, rs1951154 alternate genotype, rs7157716 reference genotype, rs7157716 alternate genotype, rs3729830 reference genotype, rs3729830 alternate genotype, rs2231126 reference genotype, rs2231126 alternate genotype, rs2231126 reference genotype, rs144420313 alternate genotype, and rs144420313 reference genotype.

In still another additional embodiment, the vector comprises a transfection reagent.

### BRIEF DESCRIPTION OF THE DRAWINGS

FIG. 1 is a conceptual illustration of an overview of an RNA therapeutic process performed by RNA therapeutic systems in accordance with embodiments of the invention.

FIG. 2 is a flowchart of a process for identifying common variants of alleles and preparing associated RNAi therapeutics in accordance with embodiments of the invention.

FIG. 3 is a flowchart of a process for applying RNAi therapeutics that target common variants of alleles in accordance with embodiments of the invention.

FIG. 4 is a data plot summarizing quantities SNPs necessary to capture the haplotype heterozygosity of human beings in accordance with embodiments of the invention.

FIG. 5 is a conceptual illustration of short read sequencing in accordance with embodiments of the invention.

FIG. 6 is a conceptual illustration of long read sequencing in accordance with embodiments of the invention.

FIG. 7 is a conceptual illustration of a long read sequencing workflow in accordance with embodiments of the invention.

FIG. 8 is a conceptual illustration of a phasing workflow in accordance with embodiments of the invention.

FIG. 9 is a data plot showing example phasing results obtained in accordance with embodiments of the invention.

FIG. 10 is a conceptual illustration of phasing results obtained in accordance with embodiments of the invention.

FIG. 11 is a conceptual illustration of phasing results obtained in accordance with embodiments of the invention.

FIG. 12 is a conceptual illustration of an experimental RNAi therapeutic in accordance with embodiments of the invention.

FIG. 13 is a conceptual illustration of an RNAi therapeutic in accordance with embodiments of the invention.

FIG. 14 is a data plot of experimental results involving an experimental RNAi therapeutic in accordance with embodiments of the invention.

FIG. 15 is a data plot of experimental results involving an experimental RNAi therapeutic in accordance with embodiments of the invention.

FIG. 16 is a sequence listing of RNAi therapeutics in accordance with embodiments of the invention.

FIG. 22 is a hardware diagram of an RNAi therapeutic server in accordance with embodiments of the invention.

FIG. 23 is a computer system diagram in accordance with embodiments of the invention.

### DETAILED DESCRIPTION

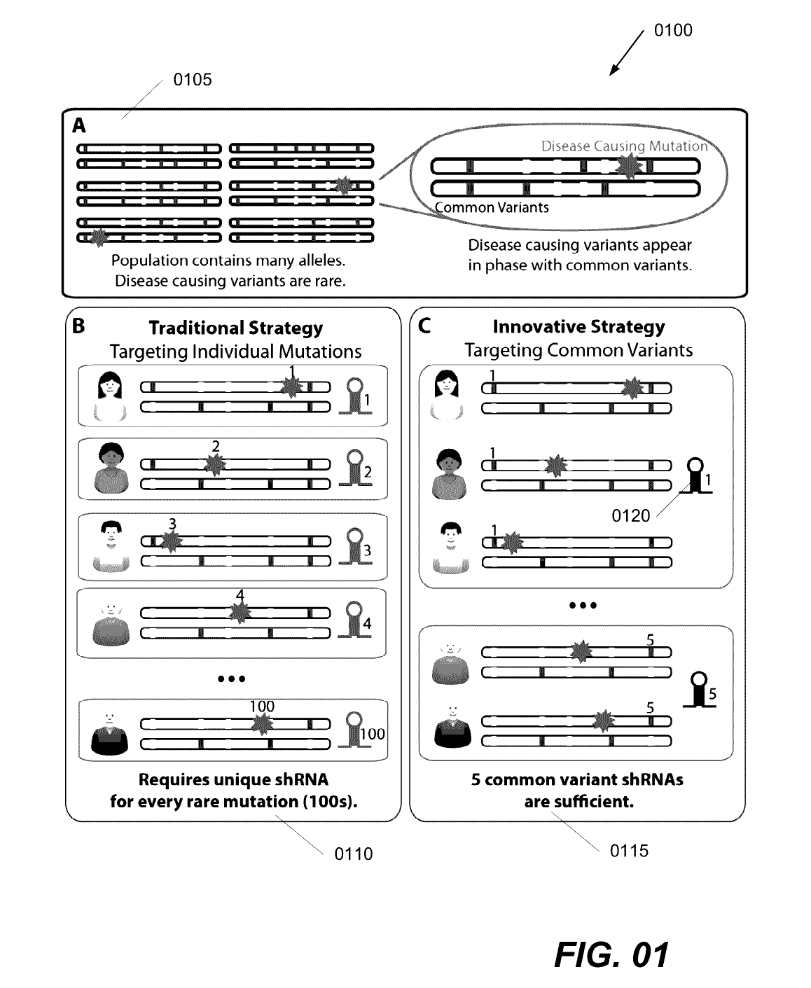

Turning now to the drawings, RNA therapeutic systems that perform processes for identifying common variants of alleles, developing RNAi therapeutics targeting the common variants, and silencing expression of deleterious alleles using RNAi therapeutics targeting the common variants in accordance with various embodiments of the invention are illustrated. FIG. 1 is a conceptual illustration of an overview 100 of an RNA therapeutic process performed by RNA therapeutic systems in accordance with embodiments of the invention. As indicated in block 105, populations of subjects contain many alleles and disease causing mutations are rare. However, common variants, namely common single nucleotide polymorphisms (SNPs), can exist within these populations. Disease causing mutations can occur in phase (on the same allele) with these common variants.

Block 110 demonstrates a traditional method of treating disease causing mutations. Each individual patient out of a representative set of 100 is individually treated with an RNAi therapeutic targeting the patient's particular mutation. This suffers from requiring a unique shRNA for every rare mutation and thus a unique therapeutic for each individual patient. Block 115 provides a conceptual illustration in accordance with many embodiments of the invention. Block 115 instead uses a single RNAi therapeutic 120 to target common variants of alleles within a group of patients. Prior methods would have to generate a new RNAi therapeutic for each patient, whereas the method illustrated in 120 is dramatically more efficient by utilizing a single RNAi therapeutic for multiple people. As noted in block 115, for certain genetically caused diseases, only five common variants within a potentially disease causing (when mutated) gene are necessary to cover the vast majority of a population.

Embodiments of the invention provide for therapeutic techniques for treating diseases caused by deleterious mutant alleles. Processes in accordance with embodiments of the invention can include diagnostic operations performed on patient samples to identify common variants of alleles on which deleterious mutations can cause diseases. The diagnostic operations can include haplotype identification using long-read sequencing techniques and computational phasing methods. Phasing, the process of determining which variants are inherited together on the same allele could identify common, benign variants that can be used as therapeutic targets. Further processes can include the selection of RNAi therapeutics targeting identified common variants on alleles. The RNAi therapeutics can include RNAi knockdown tools including shRNAs, siRNAs, and antisense oligonucleotides (ASOs). Administration of the selected therapeutics can silence the identified deleterious variants in order to treat diseases. Where a deleterious mutation is found via long read phasing on a patient sample, the deleterious mutant DNA can be silenced from expression utilizing the pre-selected RNAi therapeutics that bind to the common variant of the allele.

Allele-specific RNA interference (RNAi) therapies can be utilized by embodiments of the invention to treat patients suffering from diseases caused by heterozygous mutations. These therapies include virally-delivered shRNAs or lipid/polymer-delivered siRNAs that target mature mRNA for degradation via the classical RNAi pathway. Clinical trials of allele-specific RNA interference have started for some neurological diseases. These trials often employ Antisense Oligonucleotides (ASOs), which promote RNA degradation via RNAse H. RNase H degrades only the RNA in RNA:DNA hybrids. Thus, where a DNA-containing ASO has bound to an RNA in a cell during, RNAse H will degrade the RNA prior to its expression as a protein. Thereby, ASOs can silence expression of RNA via RNAse H.

While embodiments of the invention can be applied to various diseases across numerous types of patients, a specific application of some embodiments of the invention to treating human hypertrophic cardiomyopathy will be discussed herein. Hypertrophic cardiomyopathy (HCM) is a leading cause of early sudden cardiac death in young people and is increasingly recognized as a significant contributor to adult onset heart failure. HCM is a genetic disease inherited in an autosomal dominant fashion which results in left ventricular wall thickening. More than half of patients with familial cardiomyopathy do not have an identified genetic cause. Additionally, there are no currently approved disease-modifying therapeutics to ameliorate cardiac structural changes or improve survival. Patient-derived induced pluripotent stem cells (iPSCs) from a hypertrophic cardiomyopathy patient can be utilized as patient samples. In addition, DNA and/or RNA samples can be taken from a subject. The patient samples and/or subject samples can be amplified with custom primers and assays targeting known cardiomyopathy related genes. HCM is often caused by single, heterozygous mutations in a few cardiac genes, including MYH7. The R403Q mutation in MYH7 is one of the most studied and well-characterized HCM causing mutations. In patients with this mutation, there exists one healthy allele and one mutant R403Q allele. This one mutant allele, one healthy allele state exists across all patients with heterozygous, single nucleotide mutations. Embodiments of the invention can alleviate HCM by downregulating expression (i.e., silencing) of the mutant allele using antisense oligonucleotides (ASOs). Of note, while much of the discussion herein involves generic diseases, cells, and/or haplotyping, many embodiments focus on human diseases, human cells from human samples, and human disease gene haplotyping.

Having discussed a brief overview of the operations and functionalities RNAi therapeutic systems in accordance with many embodiments of the invention, a discussion of technical terms and a more detailed discussion of system and methods for RNAi therapeutic systems in accordance with embodiments of the invention follows below.

### DEFINITIONS OF TECHNICAL TERMS

In describing embodiments of the invention, the following terms may be employed, and are intended to be defined as indicated below. It must be noted that, as used in this specification and the appended claims, the singular forms “a”, “an” and “the” include plural referents unless the content clearly dictates otherwise. Thus, for example, reference to “an RNA” includes a mixture of two or more RNAs, and the like. In addition, where the term “set” is utilized, the set includes at least one of the entity associated the set. For instance, a “set” of alleles includes at least one allele. Of note, where a quoted technical term from the definition of technical terms is used to describe a particular embodiment of the invention herein, at least a portion of the text describing the quoted technical term can be taken to describe at least one aspect of the particular embodiment of the invention.

The term “haplotype” can be a set of DNA variations and/or polymorphisms that tend to be inherited together. A haplotype can also refer to a combination of alleles (potentially on different genes) or to a set of single nucleotide polymorphisms (SNPs) found on the same chromosome that are sufficiently closely linked to be inherited as a unit. As will be described below in connections with embodiments of the invention, relatively few sets of SNPs can capture the heterozygosity of the genetic information for large portions of human populations

The terms “RNA interference oligonucleotide”, “RNAi oligonucleotide”, “Antisense Oligonucleotides” (ASOs), and/or “RNAi therapeutic” refers to RNA and RNA-like molecules that can interact with the RNA-induced silencing complex (RISC) to guide downregulation of target transcripts based on sequence complementarity to the RNAi oligonucleotide. One strand of the RNAi oligonucleotide is incorporated into RISC, which uses this strand to identify mRNA molecules that are at least partially complementary to the incorporated RNAi oligonucleotide strand, and then cleaves these target mRNAs or inhibits their translation. The RNAi oligonucleotide strand that is incorporated into RISC is known as the guide strand and is usually the antisense strand. RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein by translational repression without cleavage of the target mRNA. Examples of RNA molecules that can interact with RISC include small interfering RNAs (siRNAs), short hairpin RNAs (shRNAs), microRNAs (miRNAs), and dicer-substrate 27-mer duplexes. The term includes RNA molecules containing one or more chemically modified nucleotides, one or more deoxyribonucleotides, and/or one or more non-phosphodiester linkages or any other RNA or RNA-like molecules that can interact with RISC and participate in RISC-mediated changes in gene expression.

The term “isoform” can be used to describe alleles of the same gene and can also refer mostly to paralogous and alternatively spliced transcripts of alleles during phasing.

As used herein, the term “small interfering RNA” or “siRNA” refers to double-stranded RNA molecules, comprising a sense strand and an antisense strand, having sufficient complementarity to one another to form a duplex. Such sense and antisense strands each have a region of complementarity ranging, for example, from about 10 to about 30 contiguous nucleotides that base pair sufficiently to form a duplex or double-stranded siRNA according to certain embodiments of the present invention. Such siRNAs are able to specifically interfere with the expression of a gene by triggering the RNAi machinery (e.g., RISC) of a cell to remove RNA transcripts having identical or homologous sequences to the siRNA sequence. As described herein, the sense and antisense strands of an siRNA may each consist of only complementary regions, or one or both strands may comprise additional sequences, including non-complementary sequences, such as 5′ or 3′ overhangs. In certain embodiments, an overhang may be of any length of nonhomologous residues, for example, 2, 3, 4, 5, 6, 7, 8, 9, 10, 11, 12, 13, 14, 15, 16 or more nucleotides. In addition, siRNAs may have other modifications, such as, for example, substituted or modified nucleotides or other sequences, which contribute to either the stability of the siRNA, its delivery to a cell or tissue, or its potency in triggering RNAi. It is to be understood that the terms “strand” and “oligonucleotide” may be used interchangeably in reference to the sense and antisense strands of siRNA compositions.

As used herein, the term “small hairpin RNA” or “shRNA” refers to an RNA sequence comprising a double-stranded stem region and a loop region at one end forming a so-called hairpin loop. In certain embodiments, the double-stranded region is typically about 19 nucleotides to about 30 nucleotides in length on each side of the stem, and the loop region is typically about three to about twelve nucleotides in length. In certain embodiments, the shRNA may include 3′- or 5′-terminal single-stranded overhangs. An overhang may be of any length of nonhomologous residues, for example, 2, 3, 4, 5, 6, 7, 8, 9, 10, 11, 12, 13, 14, 15, 16, or more nucleotides. In addition, such shRNAs may have other modifications, such as, for example, substituted or modified nucleotides or other sequences, which contribute to either the stability of the shRNA, its delivery to a cell or tissue, or its potency in triggering RNAi. In some cases, the shRNA may be derived from an siRNA, the shRNA comprising the sense strand and antisense strand of the siRNA connected by a loop

The terms “hybridize” and “hybridization” refer to the formation of complexes between nucleotide sequences which are sufficiently complementary to form complexes via Watson-Crick base pairing.

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. When a uracil is denoted in the context of the present invention, however, the ability to substitute a thymine is implied, unless otherwise stated. “Complementarity” may exist between two RNA strands, two DNA strands, or between a RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art. For purposes of RNAi, sense and antisense strands of an siRNA or sense and antisense sequences of a shRNA composition may be deemed “complementary” if they have sufficient base-pairing to form a duplex (i.e., they hybridize with each other at a physiological temperature). The antisense (guide) strand of an siRNA or shRNA directs RNA-induced silencing complex (RISC) to mRNA that has a complementary sequence.

A “target site” is the nucleic acid sequence recognized by an RNAi oligonucleotide (e.g., siRNA or shRNA). Typically, the target site is located within the coding region of a mRNA. The target site may be allele-specific (e.g., human myosin MYH7 allele encoding MHC-403Q or human MYL2 allele encoding RLC-47K).

“Administering” and/or “applying” an RNAi oligonucleotide (e.g., siRNA or shRNA) or an expression vector or nucleic acid encoding an RNAi oligonucleotide or an RNAi therapeutic to a cell comprises (but is not limited to) transducing, transfecting, electroporating, translocating, fusing, phagocytosing, shooting or ballistic methods, etc., e.g., any means by which a nucleic acid can be transported across a cell membrane.

The term “downregulating expression” refers to reduced expression of an mRNA or protein after administering or expressing an amount of an RNAi oligonucleotide (e.g., an siRNA or shRNA) and/or RNAi therapeutic. An RNAi oligonucleotide may downregulate expression, for example, by reducing translation of the target mRNA into protein, for example, through mRNA cleavage or through direct inhibition of translation. The reduction in expression of the target mRNA or the corresponding protein is commonly referred to as “knockdown” and/or “silencing”. Downregulation or knockdown of expression may be complete or partial (e.g., all expression, some expression, or most expression of the target mRNA or protein is blocked by an RNAi oligonucleotide). For example, an RNAi oligonucleotide may reduce the expression of a mRNA or protein by 25%-100%, 30%-90%, 40%-80%, 50%-75%, or any amount in between these ranges, including at least 25%, 30%, 40%, 50%, 60%, 70%, 80%, or 90%, as compared to native or control levels. Downregulation of a target mRNA or protein may be the result of administering a single RNAi oligonucleotide or multiple (i.e., two or more) RNAi oligonucleotides or vectors encoding them.

By “selectively binds” is meant that the molecule binds preferentially to the target of interest or binds with greater affinity to the target than to other molecules. For example, an RNAi oligonucleotide (e.g., siRNA or shRNA) will bind to a substantially complementary sequence and not to unrelated sequences. An oligonucleotide that “selectively binds” to a particular allele, such as a particular mutant human MYH7 or human MYL2 allele (e.g., MYH7 allele encoding MHC-403Q or MYL2 allele encoding RLC-47K), denotes an RNAi oligonucleotide (e.g., an siRNA or shRNA) that binds preferentially to the particular target allele, but to a lesser extent to a wild-type allele or other sequences. An RNAi oligonucleotide that selectively binds to a particular target mRNA will selectively downregulate expression of that target mRNA, that is, the expression of the target mRNA will be reduced to a greater extent than other mRNAs.

The term “derived from” is used herein to identify the original source of a molecule but is not meant to limit the method by which the molecule is made which can be, for example, by chemical synthesis or recombinant means.

By “isolated” when referring to a polynucleotide, such as a mRNA, RNAi oligonucleotide (e.g., siRNA or shRNA), or other nucleic acid is meant that the indicated molecule is present in the substantial absence of other biological macromolecules of the same type. Thus, an isolated siRNA or shRNA molecule refers to a polynucleotide molecule, which is substantially free of other polynucleotide molecules, e.g., other siRNA or shRNA molecules that do not target the same RNA nucleotide sequence. The molecule may, however, include some additional bases or moieties which do not deleteriously affect the basic characteristics of the composition.

“Substantially purified” generally refers to isolation of a substance (e.g., compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The terms “polynucleotide,” “oligonucleotide,” “nucleic acid,” and “nucleic acid molecule” are used herein to include a polymeric form of nucleotides of any length, either ribonucleotides or deoxyribonucleotides. These terms refer to the primary structure of the molecule. Thus, the terms include triple-, double- and single-stranded DNA, as well as triple-, double- and single-stranded RNA. Also included are modifications, such as by methylation and/or by capping, and unmodified forms of the polynucleotide. More particularly, the terms “polynucleotide,” “oligonucleotide,” “nucleic acid,” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid,” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, siRNA, shRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom).

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), cerulean fluorescent protein, Dronpa, mCherry, mOrange, mPlum, Venus, firefly luciferase, Renilla luciferase, NADPH, beta-galactosidase, horseradish peroxidase, glucose oxidase, alkaline phosphatase, chloramphenical acetyl transferase, urease, MRI contrast agents (e.g., gadodiamide, gadobenic acid, gadopentetic acid, gadoteridol, gadofosveset, gadoversetamide, and gadoxetic acid), and computed tomography (CT) contrast agents (e.g., Diatrizoic acid, Metrizoic acid, Iodamide, Iotalamic acid, Ioxitalamic acid, Ioglicic acid, Acetrizoic acid, Iocarmic acid, Methiodal, Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, and Calcium iopodate).

“Recombinant” as used herein to describe a nucleic acid molecule means a polynucleotide of genomic, RNA, siRNA, shRNA, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide.

“Recombinant host cells,” “host cells,” “cells,” “cell lines,” “cell cultures,” and other such terms denoting microorganisms or higher eukaryotic cell lines cultured as unicellular entities, refer to cells which can be, or have been, used as recipients for recombinant vector or other transferred DNA or RNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of any one or more of transcription of a mRNA or RNAi oligonucleotide, such as an siRNA or shRNA, from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

The term “transfection” is used to refer to the uptake of foreign DNA or RNA by a cell. A cell has been “transfected” when exogenous DNA or RNA has been introduced inside the cell membrane. A number of transfection techniques are generally known in the art. Such techniques can be used to introduce one or more exogenous DNA or RNA moieties into suitable host cells. The term refers to both stable and transient uptake of the genetic material, and includes uptake of an RNAi oligonucleotide (e.g., siRNA or shRNA) or an expression vector comprising an RNAi oligonucleotide. Various embodiments of the invention may utilize transfection agents, such as (but not limited to) TransIT-TKO® produced by Mirus Biosciences LLC. TransIT-TKO® is a broad spectrum siRNA transfection reagent that enables high efficiency siRNA delivery and knockdown of target gene expression in many cell types including primary cells.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contain within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

The term “3′ overhang” refers to at least one unpaired nucleotide extending out from the 3′-end of at least one strand of a duplexed RNA (e.g., double-stranded siRNA or stem region of shRNA). Similarly, the term “5′ overhang” refers to at least one unpaired nucleotide extending out from the 5′-end of at least one strand of a duplexed RNA. An overhang may be of any length of nonhomologous residues, for example, 2, 3, 4, 5, 6, 7, 8, 9, 10, 11, 12, 13, 14, 15, 16 or more nucleotides.

The term “region” when applied to polynucleotides generally refers to a contiguous portion or sequence of a single-stranded or double-stranded polynucleotide molecule. However, the term “region” may also refer to an entire single-stranded or double-stranded polynucleotide molecule.

The term “physiological conditions” refers to conditions that approximate the chemical and/or temperature environment that may exist within the body of an individual, subject, or patient.

The term “physiological temperature” generally refers to a temperature present within the body of an individual, subject, or patient. The term “physiological temperature” may be assumed to be approximately 37° C. unless otherwise specified.

The term “sense RNA” refers to an RNA sequence corresponding to all or a portion of a coding sequence of a gene or all or a portion of a plus (+) strand or mRNA sequence generated from a gene, or an RNA sequence homologous thereto.

The term “antisense strand” refers to an RNA sequence corresponding to all or a portion of a template sequence of a gene, or a sequence homologous thereto, or a minus (−) strand or all or a portion of a sequence complementary to a mRNA sequence generated from a gene.

The term “hybridize” refers to associating two complementary nucleic acid strands to form a double-stranded molecule which may contain two DNA strands, two RNA strands, one DNA and one RNA strand, etc.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

The term “about,” particularly in reference to a given quantity, is meant to encompass deviations of plus or minus five percent.

An “effective amount” of an RNAi oligonucleotide (e.g., siRNA or shRNA) or a recombinant polynucleotide or vector encoding an RNAi oligonucleotide or an RNAi therapeutic is an amount sufficient to effect beneficial or desired results, such as an amount that downregulates expression of a target mRNA or protein (e.g., human myosin MYH7 allele encoding MHC-403Q or human MYL2 allele encoding RLC-47K). For an RNAi oligonucleotide (e.g., an siRNA or shRNA), an effective amount may reduce translation or increase degradation of the mRNA targeted by the RNAi oligonucleotide. An effective amount can be administered in one or more administrations, applications or dosages.

By “therapeutically effective dose or amount” of an RNAi oligonucleotide (e.g., siRNA or shRNA) or a recombinant polynucleotide or vector encoding an RNAi oligonucleotide or an RNAi therapeutic is intended an amount that, when administered as described herein, brings about a positive therapeutic response, such as improved recovery from cardiomyopathy. Improved recovery may include a reduction in one or more cardiac symptoms, such as dyspnea, chest pain, heart palpitations, lightheadedness, or syncope. Additionally, a therapeutically effective dose or amount of an RNAi oligonucleotide or a recombinant polynucleotide or vector encoding an RNAi oligonucleotide may improve cardiomyocyte contractile strength and sarcomere alignment. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

By “subject” and/or “patient” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### Processes in Accordance with Embodiments of the Invention

RNAi therapeutics systems can perform a number of processes and/or workflows in connection RNAi therapeutics. For instance, these processes can include processes for identifying common variants of alleles, generating RNAi therapeutics targeting common variants of alleles, sequencing patient samples, phasing patient samples, and/or applying RNAi therapeutics targeting common variants of alleles. Such applied RNAi therapeutics can treat diseases caused by deleterious mutant alleles by silencing the expression of said deleterious mutant alleles. Such processes in accordance with many embodiments of the invention rely on a core efficiency gained by targeting common variants of alleles upon which mutations can cause genetically linked diseases. These common variants can each be described as a set of SNPs. Thereby, RNAi therapeutics can be designed to target the common variant and utilized across multiple patients and/or subjects without having to design individually tailored ASOs specifically for specific deleterious mutations.

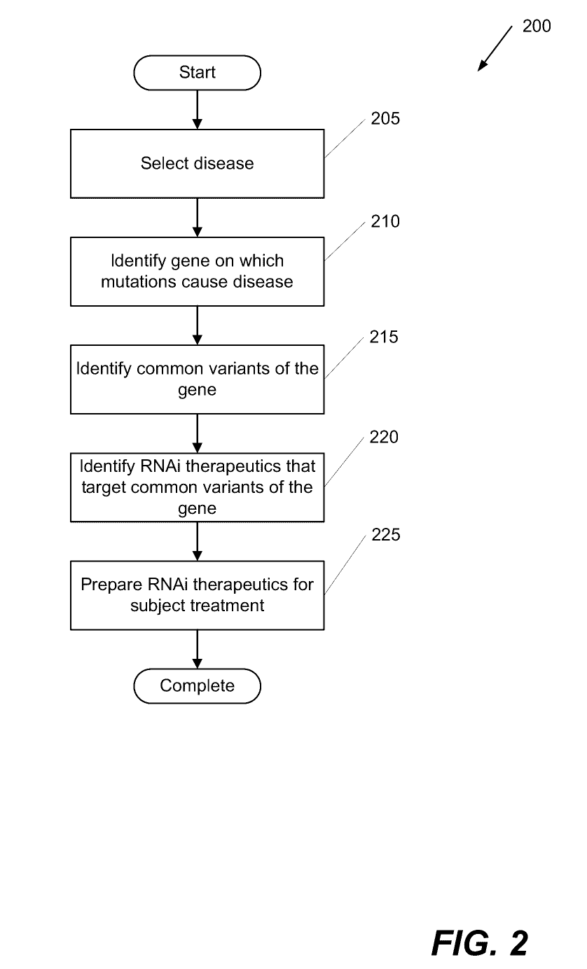

FIG. 2 conceptually illustrates a process 200 performed by RNAi therapeutics systems of in accordance with several embodiments of the invention for preparing RNAi therapeutics to target common variants of alleles. Rather than targeting deleterious subject-specific mutations, the prepared RNAi therapeutics target particular common, benign single nucleotide chance, or common variants, on the same alleles as the deleterious subject-specific mutations. Thus, the RNAi therapeutics can be used to silence disease causing alleles without having to be tailored to specific subject DNA and/or patient DNA. Process 200 can be performed by an RNAi therapeutics system in accordance with the embodiment described above in connection with FIG. 1.

Process 200 can include selecting (205) a disease. Typically, process 200 will select for genetically caused diseases that can be treated using RNAi therapeutics. Diseases can be caused by genetic mutations in the genetic information of persons. People (and other diploid organisms) have two alleles of every gene (one from mother, one from father). Genetically caused diseases can be caused by heterozygous mutations. Heterozygous mutations occur on one allele and not the other, thus leaving a person with a genetic disease having one mutated allele and one healthy allele. Well targeted and well delivered RNAi therapeutics can silence expression of the mutated allele without muting expression of the healthy allele. Various embodiments of the invention can be applied to diseases not induced by genetic mutations but still treatable by RNAi therapeutics without departing from the spirit of the invention.

Process 200 can include identifying (210) a gene on which mutations thereof cause the selected disease. Identification of the mutation carrying allele can be accomplished via various means in different embodiments. Some embodiments utilize computing systems to identifying single nucleotide or multi nucleotide polymorphisms that are associated with diseases. Alternatively, identification of an allele mutation that causes a disease can be received from another identification process. In the HCM example discussed herein, the MYH7 gene can be identified as the gene on which mutations thereof cause HCM. MYH7 is one of a number of cardiac disease associated genes. A heterozygous mutation (such as the R403Q mutation) of the MYH7 gene on one allele can cause HCM in a subject.

Process 200 can include identifying (215) common variants of the identified gene on which mutations thereof cause the selected disease. Individualized targeting of RNAi therapeutics to each subject would be extremely costly. The common variants can be identified by performing a haplotyping process (e.g., microfluidic whole genome haplotyping or long read sequencing) to physically separate individual chromosomes from a metaphase cell. This can be following by direct resolution of the haplotype. Haplotypes can include a group of genes and/or alleles inherited from one parent and are likely to be conserved between generations. Small quantities of single nucleotide polymorphisms (SNPs) in haplotypes can capture the heterozygosity of varying human populations. For instance, five common SNPs of the MYH7 gene can capture approximately 90% of the haplotype heterozygosity of all continental populations of human beings. Some embodiments target allele variants based on these five common SNPs of the MYH7 gene. As noted above, the R403Q mutation of the MYH7 gene can cause HCM in a subject. The common SNPs are not disease associated SNPs. Rather, they are simply common variants that humans can have. This commonality allows for cost efficient targeting because a set of 5-10 RNAi therapeutics can target these sets of common variants in some embodiments of the invention.

Process 200 can include identifying (220) RNAi therapeutics (i.e., allele-specific RNA interference (RNAi) therapies) that target common variants of the identified gene on which mutations thereof cause the selected disease. Various embodiments of the invention can utilize many different kinds of RNAi therapeutics as are required and/or effective for treating the selected disease. The identified RNAi therapeutics can include virally-delivered shRNAs or lipid/polymer-delivered siRNAs that target mature mRNA for degradation via the RNAi pathway. Embodiments often employ Antisense Oligonucleotides (ASOs), which promote RNA degradation via RNAse H. Specific examples of ASOs used in embodiments of the invention in treating HCM are discussed in detail below in connection with FIG. SDGM.

Process 200 also includes preparing (225) the identified RNAi therapeutics for subject treatment. The preparation can involve numerous different operations in varying embodiments; including (but not limited to) storage, purchase, labeling, and/or distribution. Preparation can also include selection and preparation of vectors. A vector is capable of transferring nucleic acid sequences (i.e., RNAi therapeutics) to target cells (e.g., viral vectors, non-viral vectors, particulate carriers, and liposomes). Typically, “vector construct,” “expression vector,” and “gene transfer vector,” can mean any nucleic acid construct capable of directing the expression of a nucleic acid of interest and which can transfer nucleic acid sequences to target cells. Thus, the term includes cloning and expression vehicles, as well as viral vectors. Additional process(es) are provided for in several embodiments for applying the prepared RNAi therapeutics when a subject afflicted with the selected disease in received.

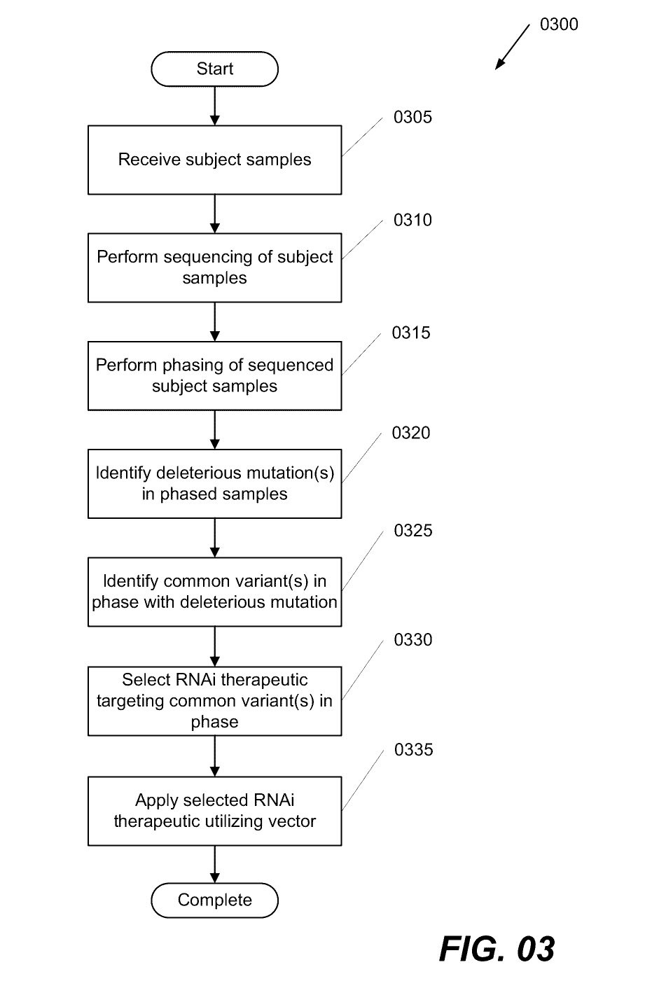

FIG. 3 conceptually illustrates a process 300 performed by RNAi therapeutics systems of in accordance with embodiments of the invention for sequencing subject samples and administering RNAi therapeutics based on common variants of alleles. Embodiments of the invention take advantage of the efficiency gained by preparing RNAi therapeutics to target common variants of alleles upon which mutations can cause diseases. Embodiments performing process 300 can leverage this efficiency to treat diseases without specifically tailoring RNAi therapeutics to the particular mutations within subject DNA. Process 300 can be performed in conjunction with, in sequence with, and/or as a part of process 200 in accordance with different embodiments of the invention.

Process 300 can include receiving (305) subject samples. The subject samples can be from a single person with a heterozygous mutation (i.e., single and/or multiple nucleotide polymorphisms) on an allele associated with a common set of variants determined in accordance with embodiments of the invention. The subject samples can also be patient samples from a patient suffering from a genetically linked disease. Various embodiments of the invention can receive the subject samples in numerous different forms; such as taken from a subject in vivo or taken from alternative sources in vitro. Process 300 can also be performed as a part of a computing system aided laboratory process where in vivo and/or in vitro samples are analyzed en masse. Often, the subject samples are taken from a patient with a known association with a particular disease. As an example, the patient may be a sufferer of HCM. Some embodiments provide for amplification of certain regions of the subject samples with custom primers. Assays can then be applied targeting known disease related genes. For instance, the assays can target cardiomyopathy (HCM) related genes such as MYH7.

Process 300 can include performing (310) sequencing of the subject samples. Sequencing enables resolution of the haplotypes of the subject(s). Embodiments of the invention can provide for various different kinds of sequencing as necessary to ascertain mutations within the Haplotypes in the subject samples. Haplotypes can include a group of genes and/or alleles inherited from one parent and are likely to be conserved between generations. Typically, embodiments utilize long read sequencing as opposed to short read sequencing. Short read sequencing usually results in sequence fragments under 200 bp. Thus, on average, each fragment captures only one or zero SNPs, making it computationally intensive to phase alleles without additional family information. Long read sequencing, which can create 10 kb or greater sequence fragments, can capture multiple SNPs per fragment, simplifying the phasing process. Long read sequencing can assist in distinguishing between different strands of DNA, which later ensures application of RNAi therapeutics to only the strands with mutated SNP(s). Sequencing can be accomplished according to workflows in accordance with some embodiments of the invention that will be described in more detail below.

Process 300 can include performing (315) phasing of the sequenced subject samples. Phasing enables distinguishing of different alleles within sequenced samples. Recognition of different phases is critical to ensure that RNAi therapeutics are selected and/or designed to target and silence mutant (i.e., deleterious) alleles without affected healthy alleles. Phasing can be accomplished according to workflows in accordance with some embodiments of the invention that will be described in more detail below. Once phased, it can be possible to resolve which strand of the subject sample contains the mutant alleles.

Process 300 can include identifying (320) deleterious mutation(s) in sequenced samples. The deleterious mutation can be polymorphism of at least one nucleotide within the subject samples. Often, the polymorphism is a heterozygous SNP on one strand of the subject sample DNA. Where the subject has HCM and it detected, the mutation potentially causing HCM can be the R403Q mutation on the MYH7 gene. Of note, many possible mutations can cause HCM, potentially hundreds or thousands of different SNP mutations of the MYH7 can induce HCM. While specific targeting schemes can be imagined, embodiments of the invention take advantage of common variants in phase with the deleterious mutation.

Process 300 can include identifying (325) common variant(s) in phase with the deleterious mutation. At least one common variant should exist on the same allele as the deleterious mutation. Thus, the deleterious mutation and the at least one common variant (i.e., common set of SNPs) should appear in at least one haplotype within the sequenced sample. Some embodiments can provide for the common variants of particular alleles to be stored previously to execution of process 300, others can perform identification of the common variants of the alleles of the sequenced sample. Typically, the subject samples are taken with knowledge of a particular disease with which to compare the samples to. For instance, where the subject has HCM, common variants of a number of cardiac-disease associated genes (e.g., the MYH7 gene) with differing sets of SNPs will be identified. In some embodiments, the identification of common variants is accomplished utilized operations from process 200 as a sub-process of process 300.

Process 300 can include selecting (330) an RNAi therapeutic targeting the at least one common variant in phase with the deleterious mutation. Where the deleterious mutation is a heterozygous deleterious mutation, the RNAi therapeutic must be selected to target the allele within the sequenced sample that possesses the deleterious mutation. The selection can be made from pre-prepared RNAi therapeutics targeting particular common variants of alleles that are subject to deleterious mutation in several embodiments. In other embodiments, RNAi therapeutic systems can synthesize and/or generate RNAi therapeutics targeting the common variant as required by process 300.

Process 300 can include applying (335) the selected RNAi therapeutic utilizing vector. The application can include mechanical and/or automated preparation of a therapeutic dose of the selected RNAi therapeutic in some embodiments. In further embodiments, the application can include administration of the selected RNAi therapeutic to a subject and/or patient by mechanical and/or human assisted means. Embodiments of the invention can utilize varying vectors as necessary to deliver a therapeutic dose of the selected RNAi therapeutic to the subject as defined above in the definition of technical terms. Process 200 and process 300 can be applied to varying diseases and subjects and/or patients without departing from the spirit of the invention. Processes in accordance with embodiments of the invention (such but not limited to processes 200 and/or 300) can be applied to numerous different diseases and/or subjects.

Although specific processes for identifying common variants of alleles, generating RNAi therapeutics targeting common variants of alleles, sequencing patient samples, phasing patient samples, and applying RNAi therapeutics targeting common variants of alleles treat mutant alleles are described above with reference to FIG. 2 and FIG. 3, combinations and sub-combinations of these processes can be utilized and even further specific operations of these processes can be executed in different orders without departing from the spirit of the invention. For instance, process 300 could be executed by an RNAi therapeutic system in accordance with a particular embodiment of the invention with portions of process 200 executed as a sub-process in order to identify a common set of variants for a particular allele associated with a particular disease. Moreover, these processes can be performed by RNAi therapeutic systems in accordance with embodiments of the invention. Examples of such RNAi therapeutic systems include (but are not limited to) the descriptions presented below in FIG. 17 and FIG. 18. In order to demonstrate the operation of processes in accordance with embodiments of the invention, several detailed examples of applications of such processes, including to subjects with HCM will be discussed below.

### Common Variants of Alleles Identified by Embodiments of the Invention

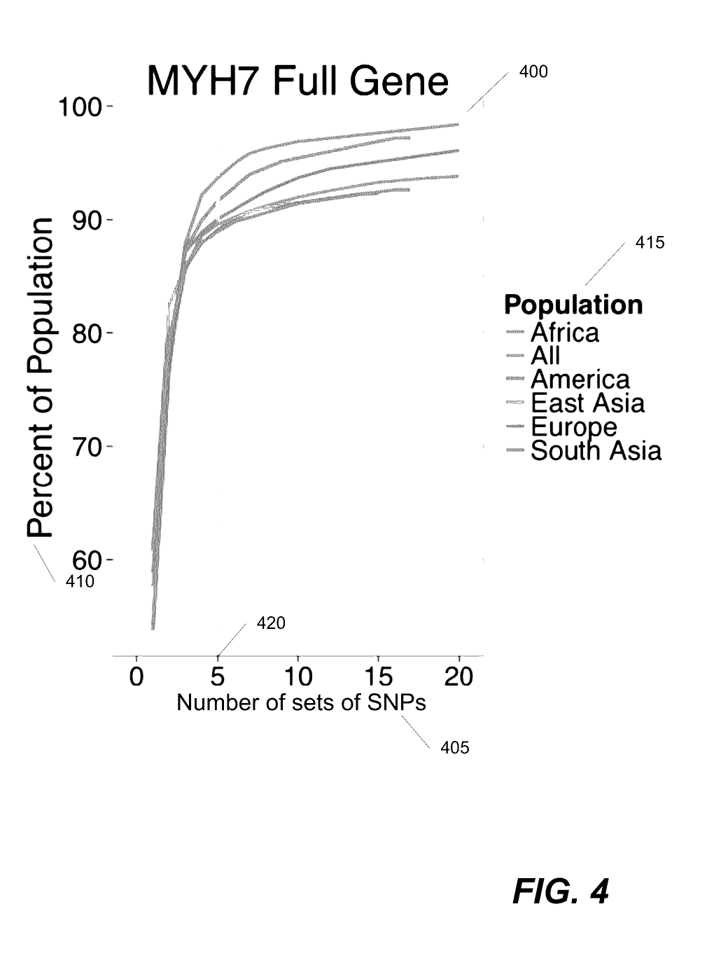

Surprisingly few common variants of alleles are necessary to capture large majorities of populations. For instance, FIG. 4 shows a data plot summarizing quantities SNPs necessary to capture the haplotype heterozygosity of human beings in accordance with embodiments of the invention. As shown, data plot 400 shows various quantities of sets of SNPs 405 necessary to capture different percentages 410 of the haplotype heterozygosity of the MYH7 gene for different human populations 415. Each set of SNPs indicated in data plot 400 can be a collection SNPs on one chromosome that tend to occur together and be inherited together. Data plot 400 shows that five sets of SNPs can capture 90% of the heterozygosity of the MYH7 gene of the listed populations. Mutations of the MYH7 gene (such as but not limited to R403Q) can be associated with HCM. The five common variants (e.g., common sets of SNPs) can be utilized as a set of common variants of the MYH7 gene by certain embodiments of the invention. In additional, data plots similar to those of 400 can be generated and/or utilized by multiple embodiments of the invention as a part of a process for identifying common variants of alleles. For instance, some embodiments of the invention performing process 200 can identify common variants of a gene (such as MYH7) by utilizing data presented in a data plot such as data plot 400.

The underlying genetic data for data plot 400 can be acquired via many different means in different embodiments. The data can be acquired from public and/or private databases of genetic and/or haplotype data. For instance, the data can be acquired from the International HapMap Project. The International HapMap Project has proposed that identifying these statistical associations and few alleles of a specific haplotype sequence can facilitate identifying all other such polymorphic sites that are nearby on the chromosome. Such information is critical for investigating the genetics of common diseases; which in fact have been investigated in humans by the International HapMap Project. The International HapMap Project provides a haplotype map (HapMap) of the human genome, which can be used to find common patterns of human genetic variation.

### Sequencing Techniques Usable by Embodiments of the Invention

DNA sequencing is the process of determining the order of nucleotides within a patient and/or subject sample. DNA sequencing can be used in determining common variants, developing targeting for RNAi therapeutics, and understanding patient samples in many embodiments of the invention. Differing embodiments of the invention can utilize either short read and/or long read sequencing.

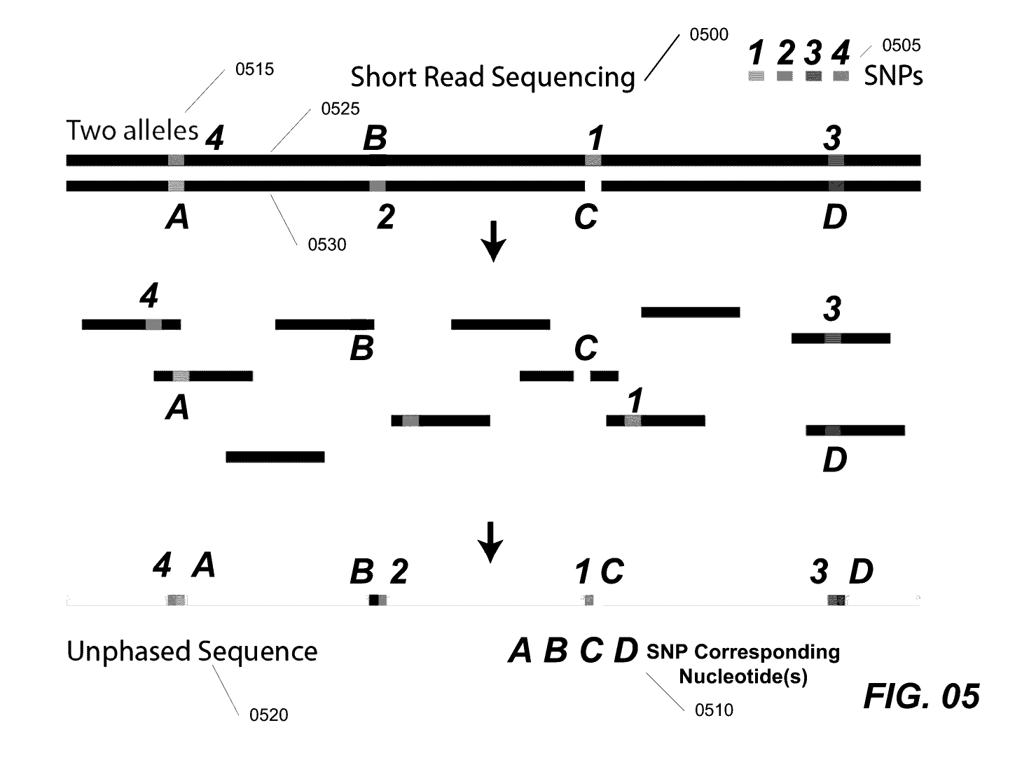

FIG. 5 is a conceptual illustration of short read sequencing in accordance with embodiments of the invention. Example short read sequencing 500 shows a conceptual illustration of two alleles 515 being sequenced. The two alleles 515 have SNPs 505 (1, 2, 3, 4) and Reference nucleotides 510 (A, B, C, D). Short read sequencing has inherent limitations when attempting to resolve the phase of portions of alleles. Short read sequencing usually results in sequence fragments under 200 base pairs. Thus, on average, each fragment captures only one or zero SNPs, making it computationally intensive to phase alleles without additional family information. Example fragments are shown between two alleles 515 and unphased sequence 520. As shown, SNPs 505 (1, 2, 3, 4) and Reference nucleotides 510 (A, B, C, D) failed to be phased in unphased sequence 520. As a result, SNPs 505 (1, 2, 3, 4) and Reference nucleotides 510 (A, B, C, D) cannot be phased to exclusively either of first allele 525 or second allele 530. Many embodiments of the invention instead utilize long read phasing.

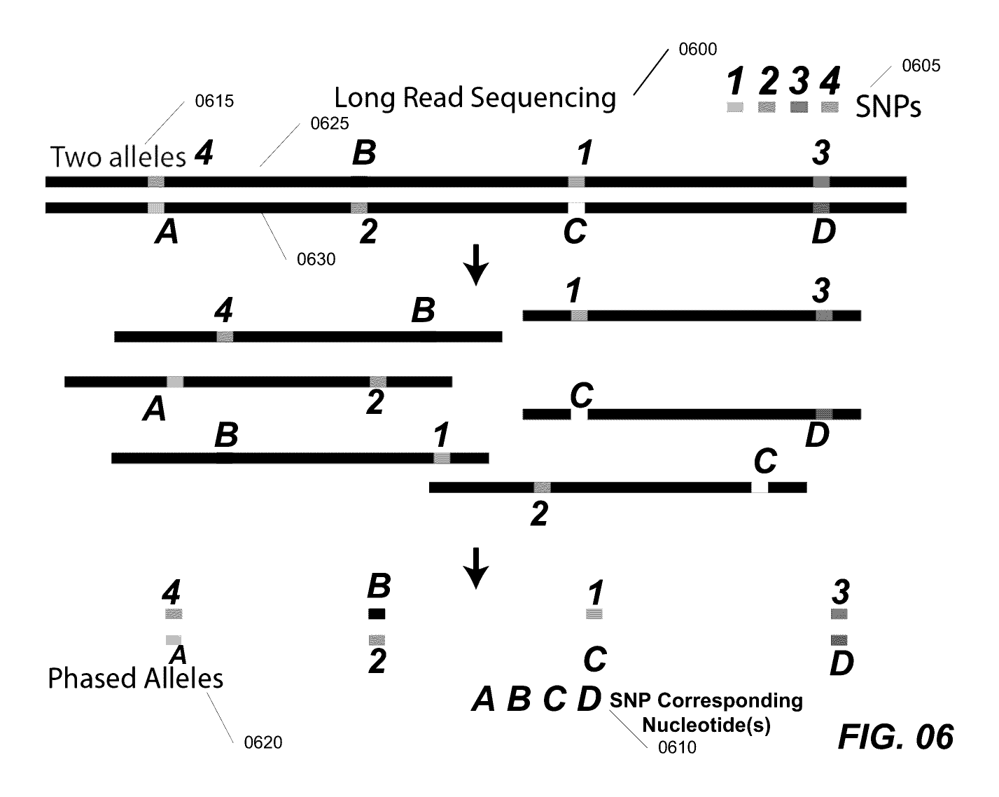

FIG. 6 is a conceptual illustration of long read sequencing in accordance with embodiments of the invention. Example long read sequencing 600 shows a conceptual illustration of two alleles 615 being sequenced. The two alleles 615 have SNPs 605 (1, 2, 3, 4) and Reference nucleotides 610 (A, B, C, D). Long read sequencing can generate 10 kilo-base-pair or greater sequence fragments and can capture multiple SNPs per fragment; thus, simplifying the phasing process. Example long read sequence fragments are shown between two alleles 615 and phased alleles 620. As shown, SNPs 605 (1, 2, 3, 4) and Reference nucleotides 610 (A, B, C, D) are correctly phased into two discrete resulting alleles 620. As a result, SNPs 605 (1, 2, 3, 4) and Reference nucleotides 610 (A, B, C, D) can be phased to exclusively either of first allele 625 or second allele 630. Many embodiments of the invention utilize long read phasing. For instance, multiple embodiments implementing operations from process 200 and 300 utilize long read sequencing. However, other embodiments may also utilize short read sequencing in conjunction with long read sequencing without departing from the spirit of the invention.

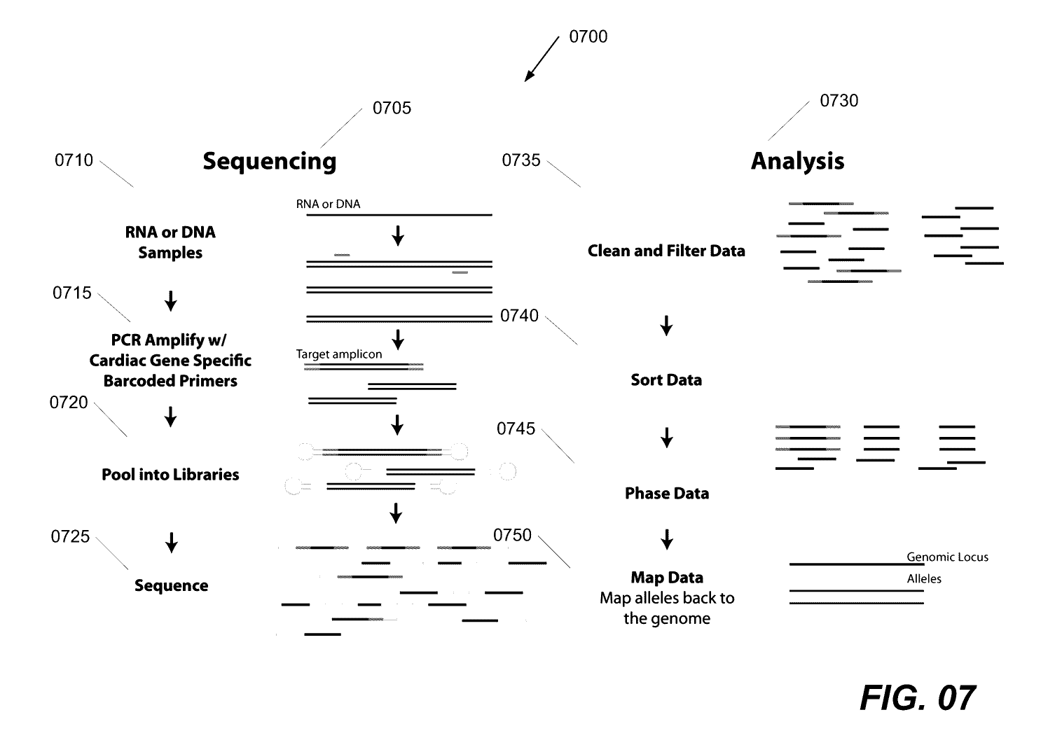

FIG. 7 is a conceptual illustration of a long read sequencing workflow in accordance with embodiments of the invention. Workflow 700 accomplishes long read sequencing of subject samples in many embodiments. Workflow 700 can be applied to subject cells for DNA sequencing in order to find deleterious mutations and common variants. In a particular example, workflow 700 can be applied to heart samples to prepare full-length sequences of MYH7 and/or MYBPC3 genes. Full-length sequencing can enable distinguishing of healthy alleles from mutant alleles that contain disease associated SNPs. Workflow 700 can be implemented as a part of a process in accordance with many embodiments of the invention. For instance, workflow 700 can be utilized during sequencing operations of processes 200 and/or 300. Workflow 700 provides for two processes for targeted RNA, cDNA and/or DNA sequencing using long read sequencing. Specifically, workflow 700 can include a sequencing process 705 and an analysis process 730.

Sequencing process 705 can include gathering (710) samples. The samples can include (but are not limited to) DNA and/or RNA. The DNA and/or RNA can include (but are not limited to), controls, point mutations, and/or splice variants. In some embodiments, the samples can be from the left ventricle of a heart. Where the samples are collected in vitro for experimental purposes, some embodiments can utilize patient-derived induced pluripotent stem cells (iPSCs) from a hypertrophic cardiomyopathy patient can be utilized as patient samples.

Sequencing process 705 can include amplifying (715) target genes within samples with barcoded primers. The amplification can be accomplished using polymerase chain reaction (PCR). PCR amplification involves heating strands of DNA double helix sufficiently to separate the strands and utilizing the cooled and separated strands as templates for DNA polymerase to selectively amplify the target DNA. The patient samples can be amplified with custom primers and assays targeting known cardiomyopathy related genes such as (but not limited to) MYH7 and MYBPC3.

Sequencing process 705 can include pooling (720) the amplified samples in libraries. This can be accomplished by adding adapters (e.g., polymerase) to the samples to circularize the samples as illustrated in FIG. 7.

Sequencing process 705 can include sequencing (725) the pooled samples. Workflow 700 can be substantially and/or entirely automated via servers and computing systems in accordance with various embodiments of the invention. The sequencing operation of a number of embodiments can utilize a number of single molecule real time sequencing (SMRT) cells with wells and waveguides. SMRT cells can enable illuminated observation that is sufficiently high resolution to observe only a single nucleotide of DNA being incorporated by DNA polymerase.

Analysis process 730 can include classifying (735) sequencing data. Classifying sequencing data can include (but is not limited to), in various embodiments, trimming adapters, finding primers, full length reads, and/or non full length reads. Analysis process 730 can include sorting (740) sequencing data. Sorting the sequencing data can be sorted according to various types of sorts including (but not limited to) separating the sequencing data by library and/or primer. Analysis process 730 can include clustering (745) sequencing data according to full length reads by isoform.

Analysis process 730 can include mapping (750) sequencing data. The mapping can be mapped by isoforms back to the genome of the samples. The isoforms have at least nearly the same nucleotide base sequences as the genome, but due to sequencing splicing are out of order as indicated in FIG. 7. The mapping can also include filtering the isoforms for coverage (e.g., greater than 99% coverage) and identity (e.g., greater than 95% identity). Where isoforms with read counts less than five are present, they can be discarded. Isoform sequences can be required to being and end with designed primer sequences.

Workflow 700, sequencing process 705, and/or analysis process 730 can be performed as a sub-process of a larger process and/or processes performed by RNAi therapeutic systems in accordance with embodiments of the invention. Specifically, sequencing process 705, and/or analysis process 730 can be performed in conjunction with process 200 shown in FIG. 2 for preparing RNAi therapeutics to target common variants of alleles and/or with process 300 for preparing RNAi therapeutics to target common variants of alleles. In addition, particular operations described above in conjunction with process 200 and/or process 300 can be included in other processes described herein in orders as necessary to implement various embodiments of the invention.

### Phasing Techniques Usable by Embodiments of the Invention

Phasing enables distinguishing of different alleles within sequenced samples. Recognition of different phases is critical to ensure that RNAi therapeutics are selected and/or designed to target and silence mutant (i.e., deleterious) alleles without affected healthy alleles. The following discussion presents several diagrams showing phasing techniques utilized by several embodiments of the invention.

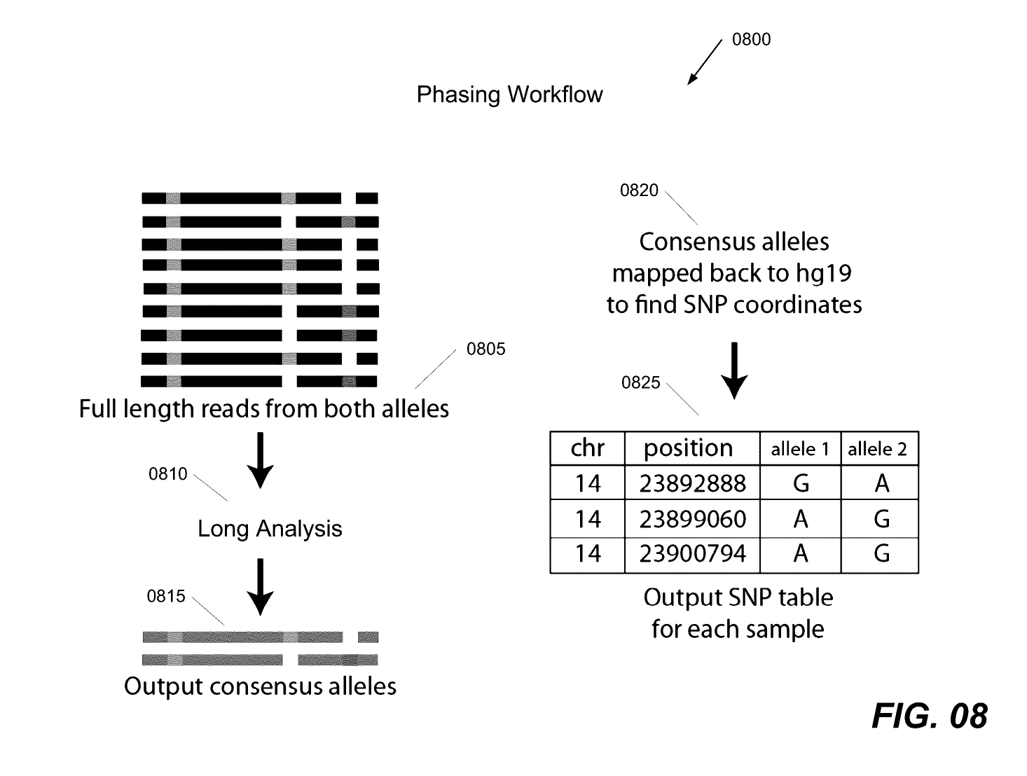

FIG. 8 is a conceptual illustration of a phasing workflow in accordance with embodiments of the invention. Phasing workflow 800 can be implemented as a process and/or sub-process of the other processes described herein in some embodiments of the invention. Phasing workflow 800 provides techniques for phasing sequenced alleles.

Phasing workflow 800 includes full length reads from both alleles of a subject sample (805). The full length reads can be produced according to sequencing workflow 700. Each full length read of a sequenced allele belongs to an isoform, as determined by sequencing workflow 700.

Phasing workflow 800 includes long analysis (810). The long analysis can be performed using the Long Amplicon Analysis (LAA) tool from PacBio SMRTAnalysis software in some embodiments. The full length reads produced by sequencing workflow 700 can be added to a whitelist and given as input to long analysis. Phasing workflow 800 can include outputting as a result of long analysis a set of consensus alleles (815).

Phasing workflow 800 includes mapping (820) consensus alleles back onto a human genome to find SNP coordinates. A table of output SNP coordinates for each sample is shown at 825. Some embodiments include discarding SNPs output by phasing workflow 800 where the SNPs are from below a threshold quantity of isoforms (for instance, discarding outputted SNPs with support from only one isoform).

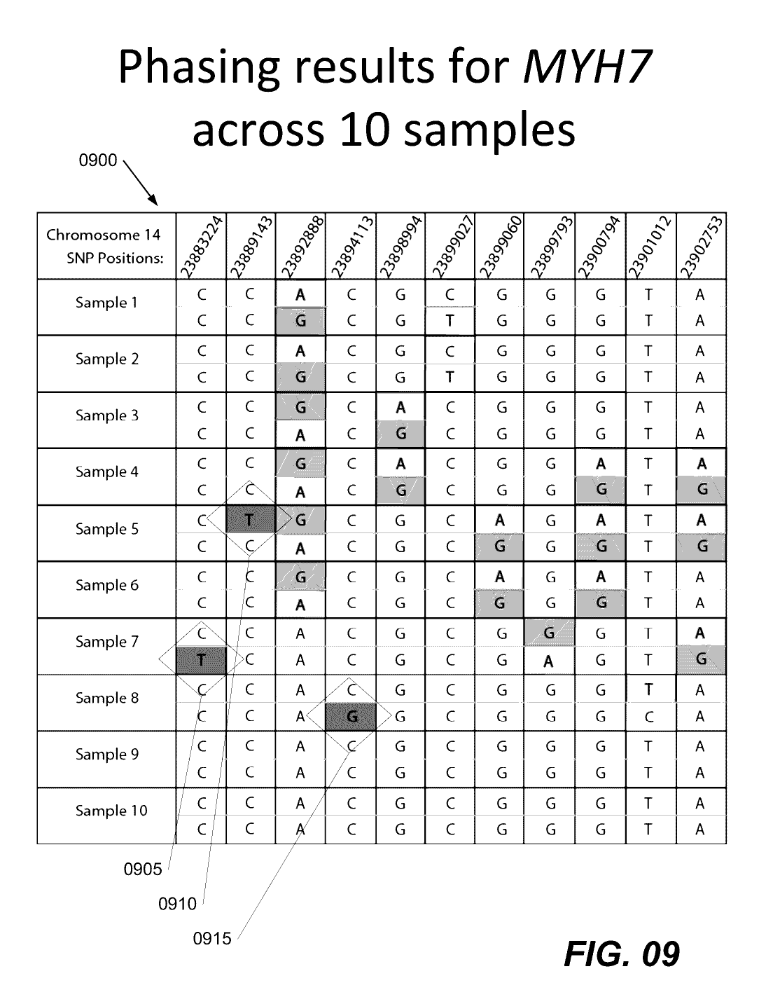

FIG. 9 is a data plot showing example phasing results obtained in accordance with embodiments of the invention. FIG. 9 shows phasing results for MYH7 on across 10 samples. As indicated in table 900, 10 samples are shown on the left hand side with 10 SNP positions on chromosome 14. The 10 samples are phased alleles from sequencing samples. Heterozygous SNPs 905 in sample 7, 910 in sample 5, and 915 in sample 9 are highlighted and called out. Samples 5, 7, and 8 contained known disease causing mutations. The mutations can occur on only one strand of each sample. These mutations were validated by techniques involving the sequencing and phasing workflows in accordance with some embodiments of the invention.

The phasing workflows have enabled certain embodiments to create haplotypes for each allele in control and disease samples. Several A-G pairs and T-C pairs are also indicated in table 900. These correspond to common sets of variants identified using processes in accordance with embodiments of the invention. The A-G pairs and T-C pairs are highlights and indicated common, relatively benign, SNPs. These common variants enable targeting with RNAi therapeutics on a smaller set of matching RNAi therapeutics without having to generate RNAi therapeutics that target the particular, rare, and deleterious Heterozygous SNPs 905 in sample 7, 910 in sample 5, and 915 in sample 9.

By way of example, to treat the patient/subject associated with sample 5, an RNAi therapeutic (e.g., an ASO) that targets the common variant G in Sample 5 at SNP position 23892888 (i.e., the third column G). The ASO would silence expression of the top strand of sample 5, thereby stopping expression of deleterious mutation T 910 at position 23889143 (i.e., the second column T). Critically, the common variant G in Sample 5 at SNP position 23892888 (i.e., the third column G) is in 6 out 10 of the samples (i.e., samples 1-6). This means that common variant G in Sample 5 at SNP position 23892888 (i.e., the third column G) occurs in 60% of the samples, making it a good candidate for targeting. Of note, the correct RNAi therapeutic must be selected to target the allele (i.e., strand) with the deleterious mutation. Suppressing both alleles can harm a live patient. Many embodiments of the invention provide for processes, sequencing workflows, and phasing workflows to ensure the correct RNAi therapeutic is chosen.

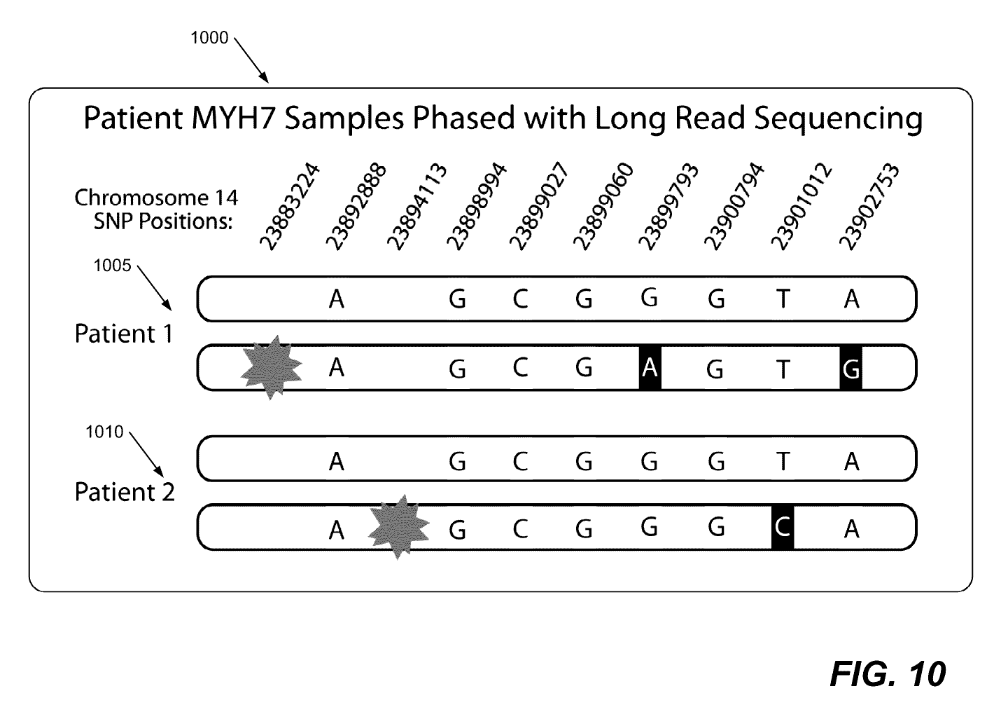

FIG. 9 is only one exemplary result from a single embodiment of the invention with respect to the MYH7 gene and HCM. MYBPC3 gene data has been similarly phased utilizing techniques according to certain embodiments of the invention. Various embodiments can produce differing phasing results from different samples, subjects, diseases, and/or genes. FIG. 10 is a different representation of FIG. 9 whereby patient 1 1005 from table 1000 corresponds to sample 7 from table 900 and patient 2 1010 from table 1000 corresponds to sample 8 from table 900.

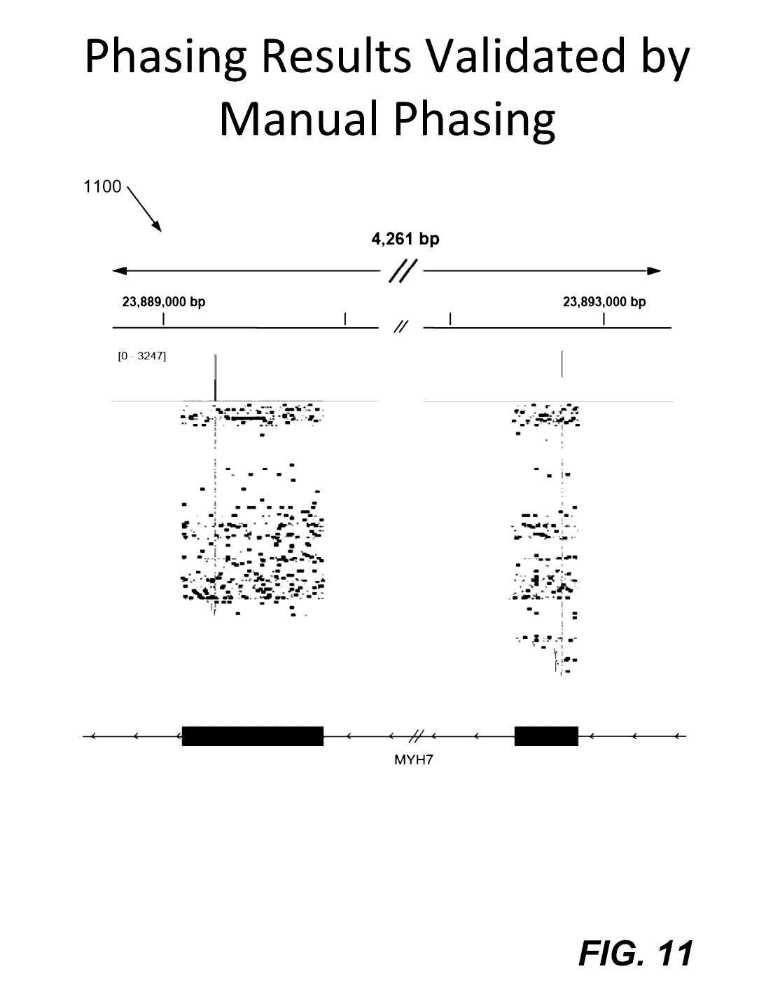

As mentioned above, phasing results from FIG. 9 and FIG. 10 have been experimentally validated. FIG. 11 depicts phasing results as validated by manual phasing. To validate the computational phasing methods employed by many embodiments of the invention, BAM files were analyzed in IGV. A BAM file (.bam) is the binary version of a SAM file. A SAM file (.sam) is a tab-delimited text file that contains sequence alignment data. Integrative Genomics Viewer (IGV) is a high-performance visualization tool for interactive exploration of large, integrated genomic datasets. As an example, shown in results 1100 are two SNPs from Sample 5 of table 900, a disease-causing mutation and a benign SNP. Phased by eye, the allele haplotypes matched computational output generated according to implementations of some embodiments of the invention.

### RNAi Therapeutic Design

One of the core challenges with RNAi therapeutics is effective delivery of the silencing molecules. Embodiments of the invention can utilize many different kinds of vectors to deliver silencing molecules. Many embodiments rely on a common variant allele-specific mutation targeting ASOs. In order to illustrate examples of such an RNAi therapeutic ASOs, the following passages will describe several experimental designs and results from applying them.

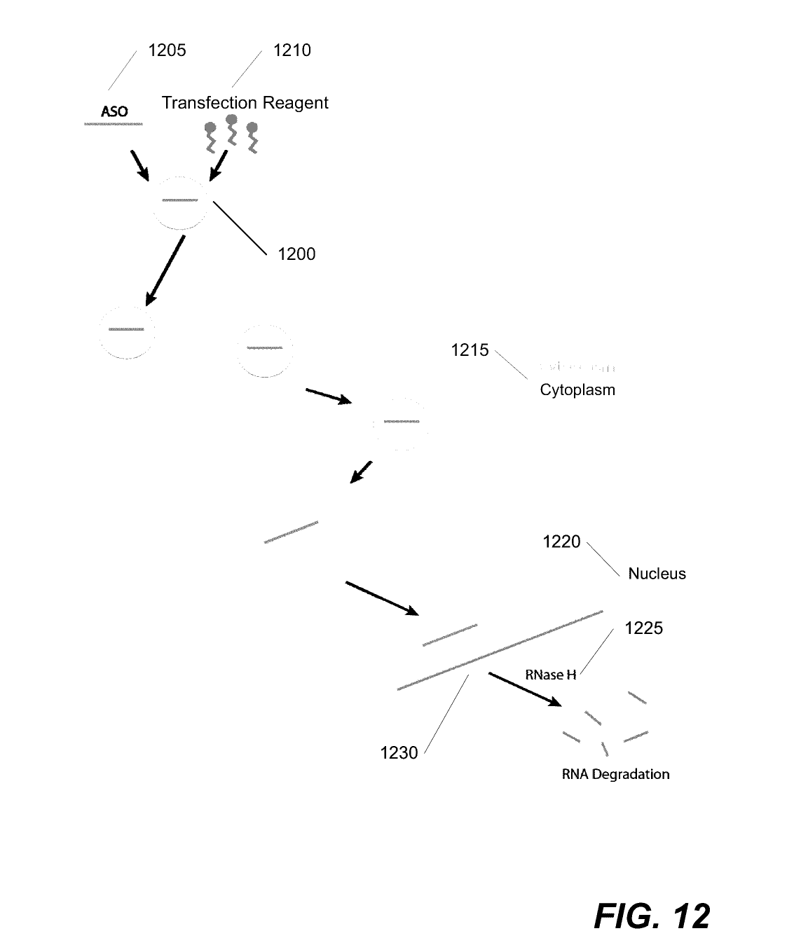

FIG. 12 is a conceptual illustration of an experimental RNAi therapeutic in accordance with embodiments of the invention. Experimental RNAi therapeutic design 1200 is shown in FIG. 12. RNAi therapeutic 1200 includes ASO 1205 and transfection reagent 1210. ASO 1205 (antisense oligonucleotides) is targeted to a common variant that can be used a targeting proxy for a deleterious mutation. Transfection reagent 1210 can be a broad spectrum siRNA transfection reagent that enables siRNA/ASO delivery and knockdown of target gene expression in cells. As shown, Experimental RNAi therapeutic design 1200 can pass through the cytoplasm 1215 and enter the nucleus 1220. The payload ASO 1205 can then bind to RNA 1230. RNase H 1225 will then degrade the RNA portions of the DNA/RNA hybrid (i.e., the DNA ASO 1205 hybridized with the RNA 1230. This results in RNA degradation, as shown in FIG. 12. Results consistent with the conceptual illustration in FIG. 12 can be (and were) achieved utilizing several embodiments of the invention over a 20-22 day old iPSC-CMs, 2 uM ASOs, a transfection reagent, and a 24 hour incubation when applied to the MYH7 gene targeting patient samples with the 403Q (HCM) mutation. Experimental RNAi therapeutic design 1200 is presented as an example.

Various embodiments of the invention can utilize many different kinds of vectors (besides or in addition to) transfection reagents. For instance, different embodiments can utilize any vector capable of transferring nucleic acid sequences to target cells (e.g., viral vectors, non-viral vectors, particulate carriers, liposomes, and cloning and expression vehicles). In addition, only one example of targeting is shown in experimental RNAi therapeutic design 1200. Various different common variants can be targeted using the principles demonstrated in experimental RNAi therapeutic design 1200 without departing from the spirit of the invention.

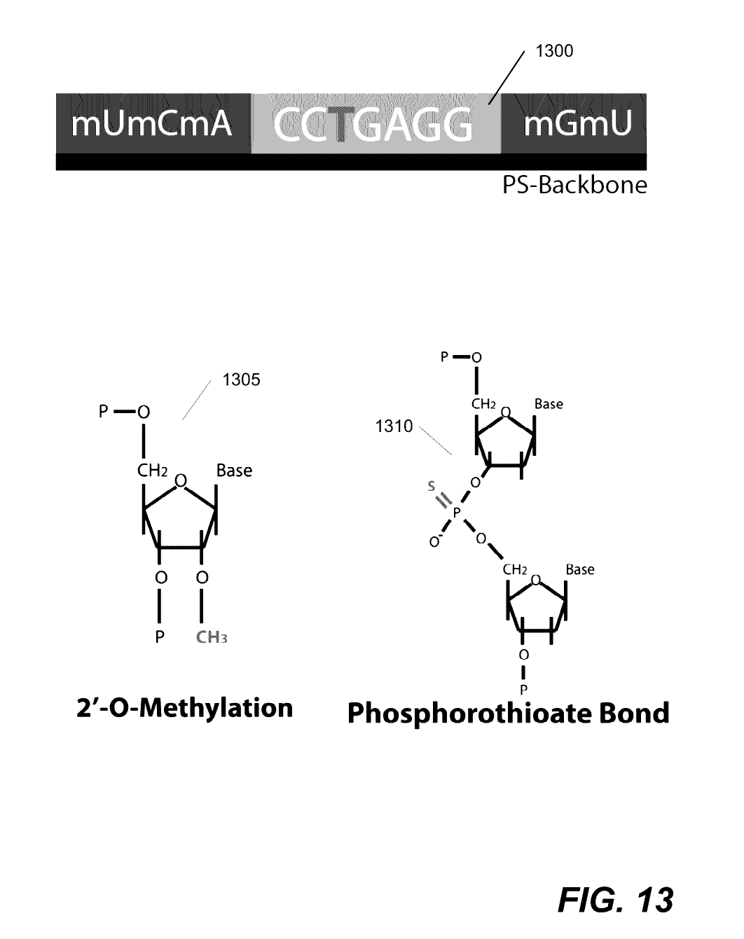

FIG. 13 is a further conceptual illustration of an RNAi therapeutic experimental design in accordance with embodiments of the invention. Experimental RNAi therapeutic design 1300 includes several DNA and RNA sections. Experimental RNAi therapeutic design 1300 includes RNA sections “mUmCmA” and “mGmU” as wings. Experimental RNAi therapeutic design 1300 includes “CCTGAGG” as DNA. Experimental RNAi therapeutic design 1300 can be referred to as a wingmer.

One of the key challenges in RNAi therapeutics is preventing the RNAi therapeutic from simply breaking down over time within and without of target cells. 2′-O-methylation 1305 gives ASOs degradation resistance. Degradation resistance can further be improved in some embodiments by substituting sulfur for non-bridging oxygen to produce phosphorothioate bond 1310. A phosphorothioate bond 1310 assists in stabilizing the RNAi therapeutic to last longer and more effectively deliver the payload ASO in some embodiments. Phosphorothioate bond 1310 can reduce attacks by endonuclease and can help the ASO get into cell via binding to cell-surface proteins. The experimental designs presented in FIG. 12 and FIG. 13 are merely illustrative, other embodiments may utilize other designs without departing from the spirit of the invention.

### Experimental RNAi Therapeutic Results

The following section discusses empirical results from the application of experimental RNAi therapeutic designs in accordance with several embodiments of the invention. These results show an improvement over the state of the art in experimental RNAi therapeutic designs and demonstrate the value of the novel and non-obvious techniques, processes, workflows, and designs described herein.

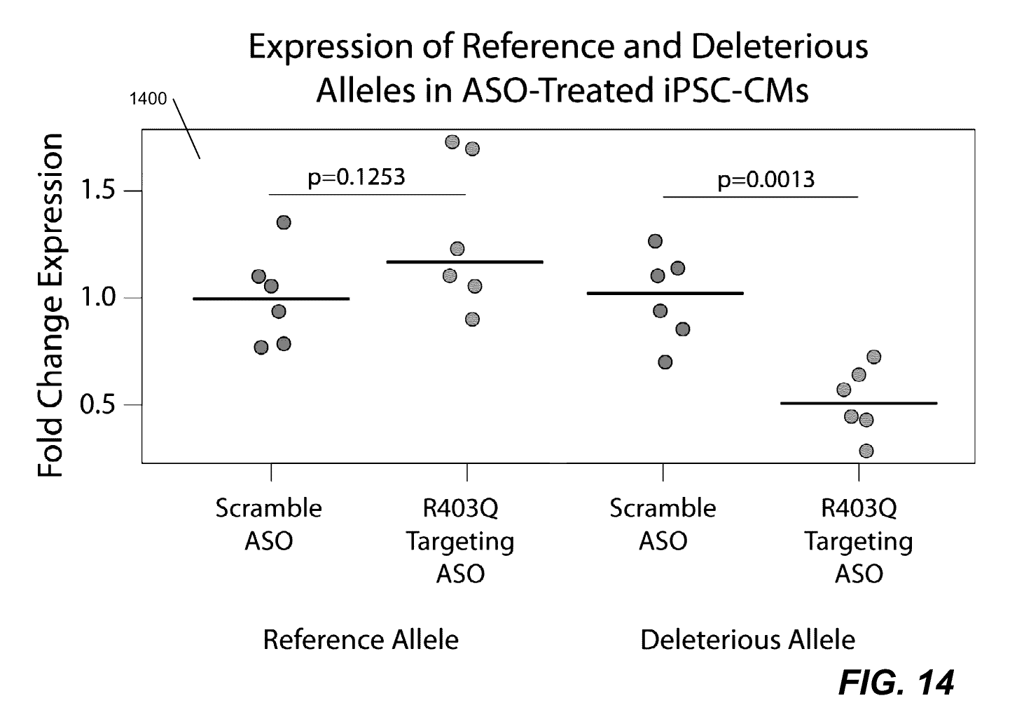

FIG. 14 is a data plot of experimental results involving an experimental RNAi therapeutic in accordance with embodiments of the invention. The experimental RNAi therapeutic was applied to reference alleles with a Scramble ASO and an R403Q Targeting ASO. Table 1400 shows a low p-value test for reference alleles. The Scramble ASO had the same level of expression (indicating no binding of the Scramble ASO to free RNA, thus no silencing effect). The R403Q targeting ASO showed expression of the reference allele that was not significantly different from that of the Scrambled ASO, indicating that it did not silence the reference allele. The R403Q targeting ASO on the reference allele had increased expression, which is not significant as indicated by the p-value. This means there was not increased expression and it did not silence the reference allele.

When targeting the deleterious allele, the Scramble ASO had the same level of expression (indicating no binding of the Scramble ASO to free RNA, thus no silencing effect). However, the R403Q Targeting ASO massively decreased expression of the deleterious allele, as indicated by the low scatter plot values in the fourth column. This experiment had a very significant p-value of p=0.0013, thus indicating it was very unlikely that such a result could have arisen from mere happenstance.

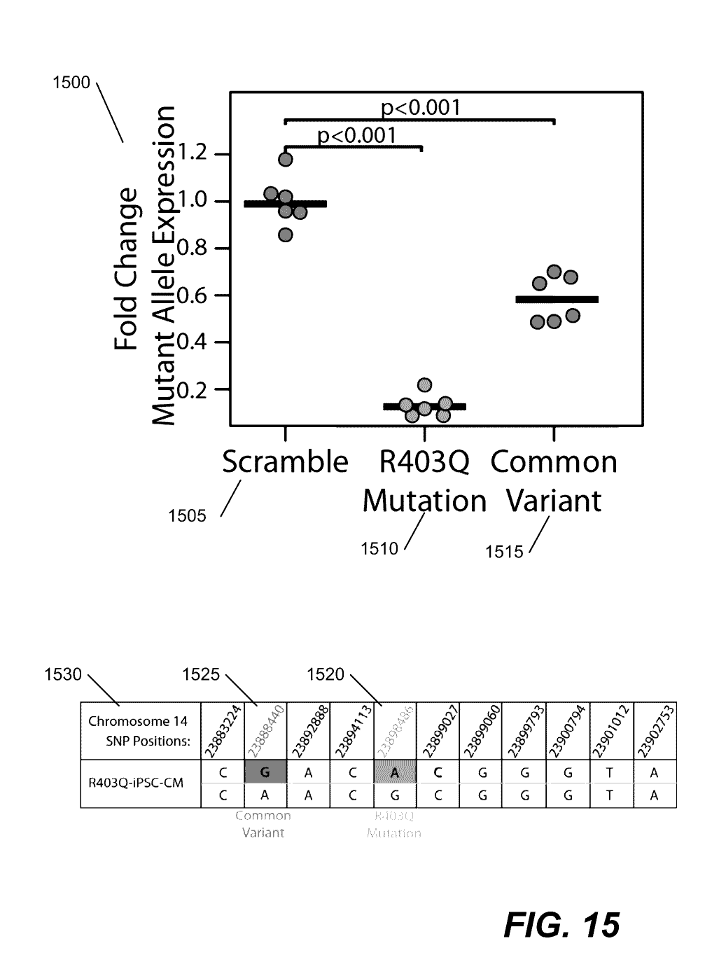

FIG. 15 is another data plot of experimental results involving an experimental RNAi therapeutic in accordance with embodiments of the invention with respect to mutant alleles (e.g., a mutant R403Q allele). The mutant allele carries a deleterious mutation and a benign common variant. The mutant alleles are the same across each experiment. The experiments include applying experimental RNAi therapeutics to the same mutant alleles.

The experimental RNAi therapeutics include the following ASOs: scrambled ASO 1505 (should not bind), an R403Q targeting ASO 1510 (should knock down mutant allele), and a common variant targeting ASO 1515 (should knock down mutant allele). As shown in table 1500, the scrambled ASO 1505 did not meaningfully affect expression of the mutant allele. The R403Q targeting ASO 1510, with its narrow targeting, severely decreased expression of the mutant allele. The common variant targeting ASO 1515, which targets a common variant on the mutant allele, significantly decreased expression of the mutant allele.

In this experiment, the common variant targeting ASO 1515 was targeting a common exonic variant (rs144420313) that lies on the same allele as the R403Q mutation. Specifically: ASO=mGmGmUmGm UAGGTGAGCTT mGmCmCmUmC. “mGmGmUmGm” was the first wing, UAGGTGAGCTT the complementary DNA, and “mGmCmCmUmC” the second wing. Further results and experimentation in the spirit of the invention will result in improved targeting discrimination over time.

### Sequences and Common Variants

Specific sequences and common variants have been shown to be utilizable for treatment of deleterious mutations for many embodiments of the invention. The following figures include tables of sequences targeting common variants of certain genes that can be utilized by some embodiments of the invention.

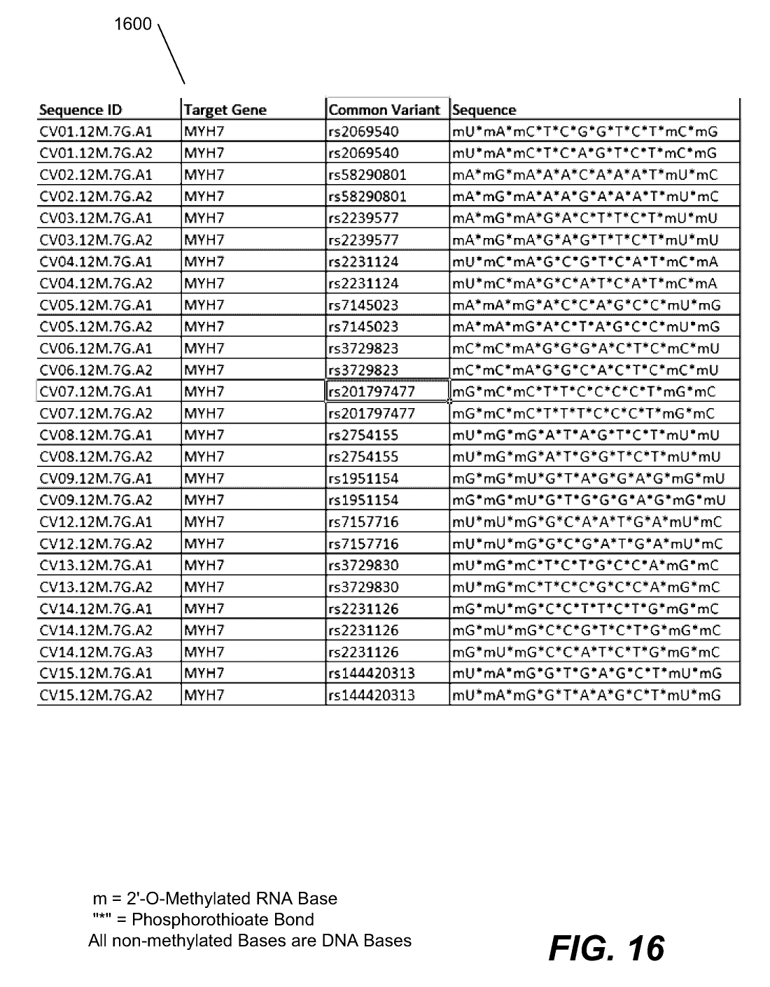

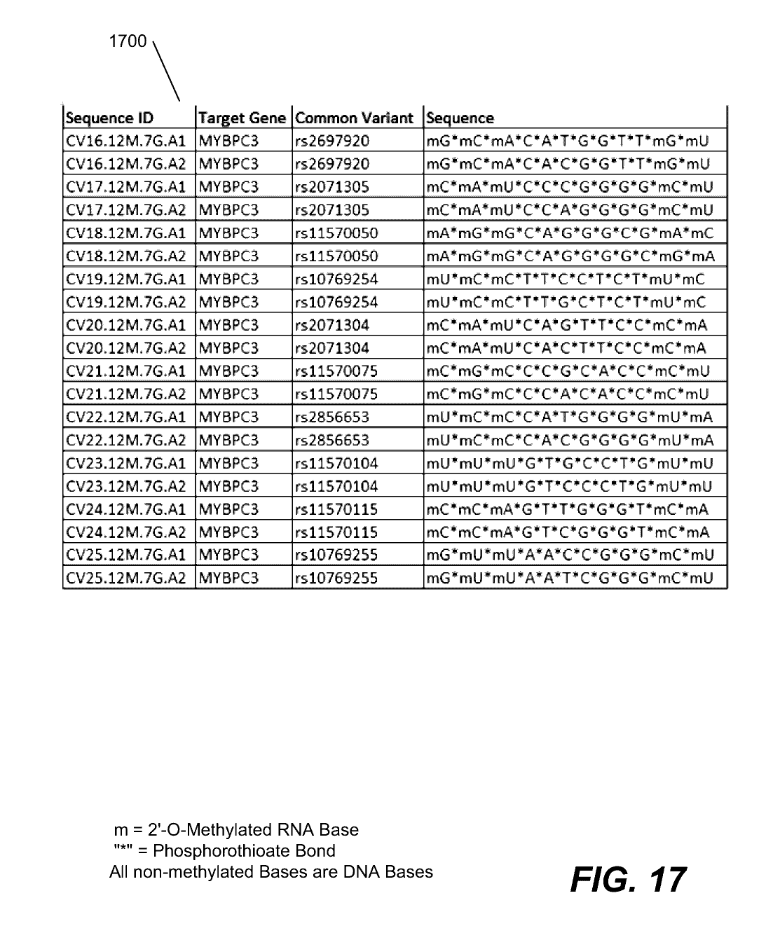

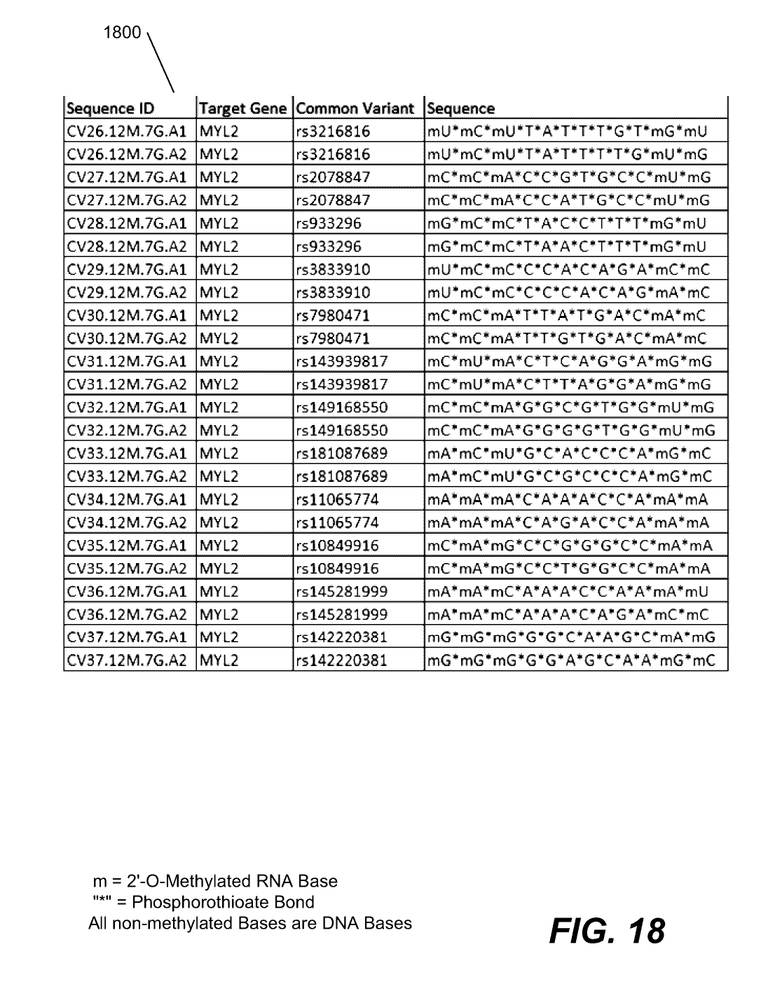

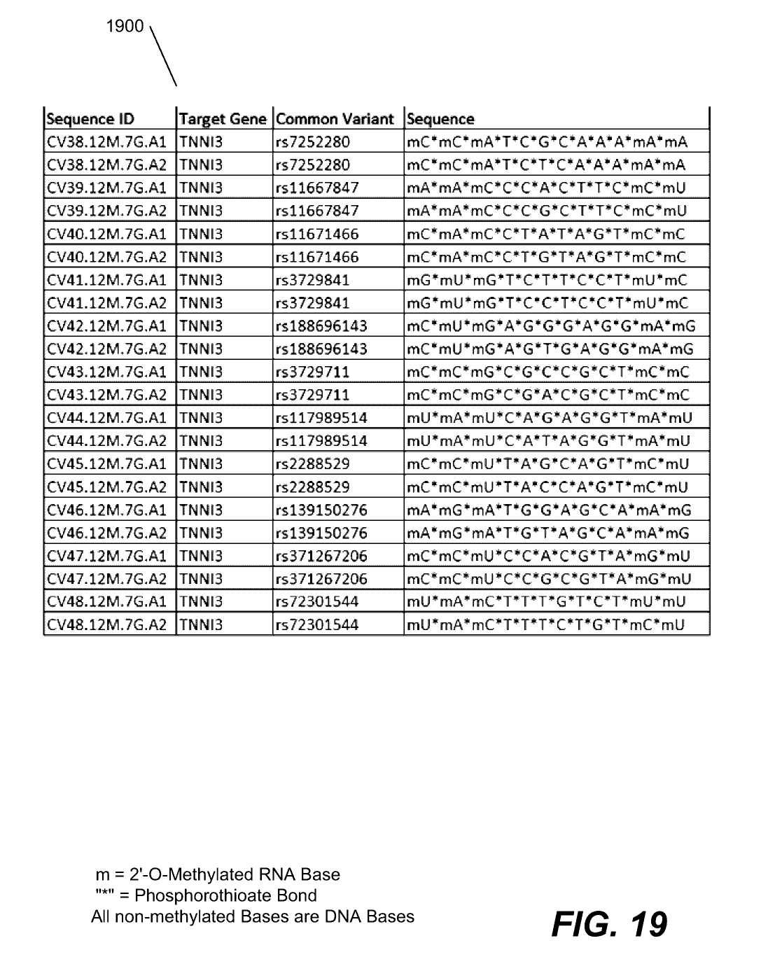

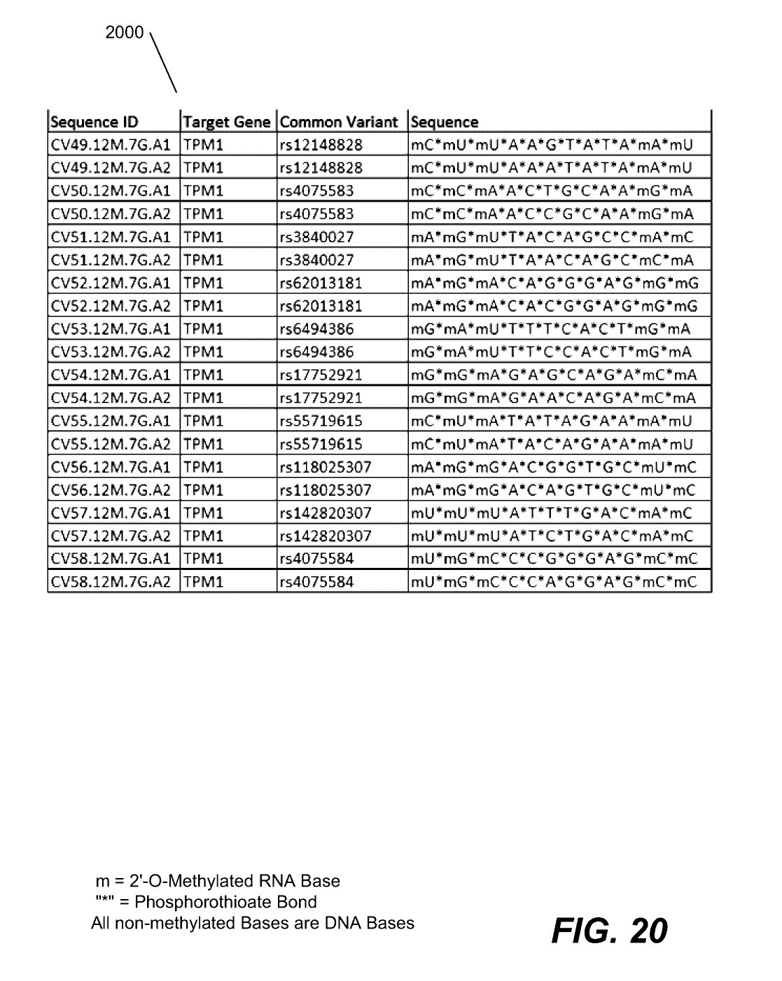

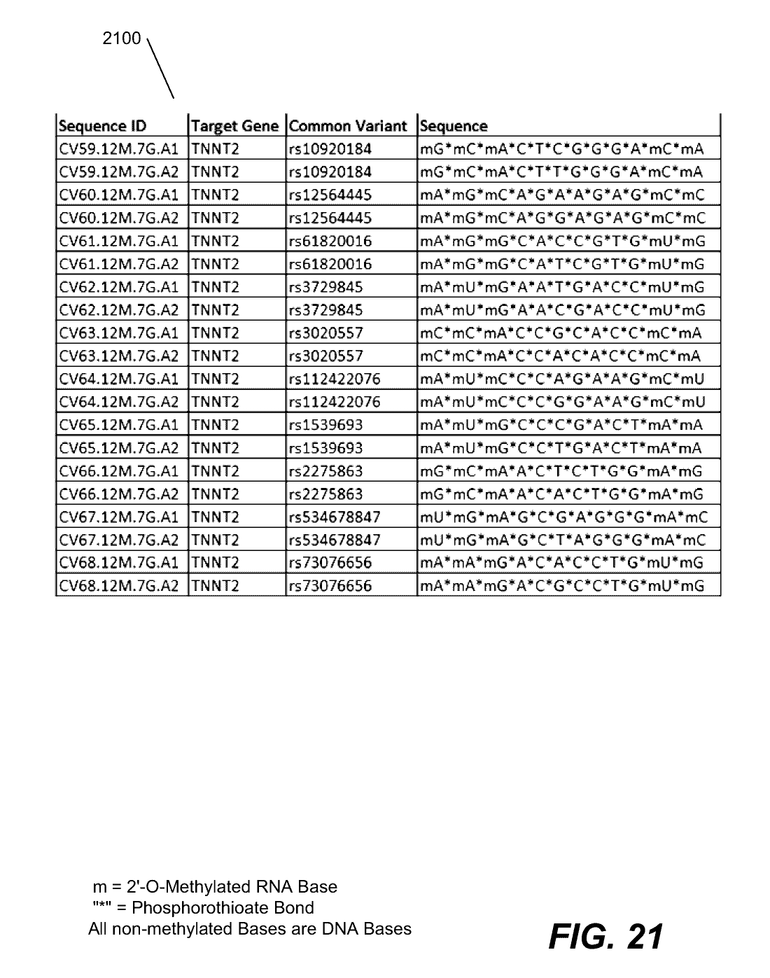

FIG. 16 provides an example sequence listings for RNAi therapeutics (i.e., ASOs) implemented and experimented with according to several embodiments of the invention. These listings provide for means of targeting common variants of deleterious alleles and offer a promise of improved treatment of genetic disorders. As shown, sequence listing 1600 includes a list of sequence IDs, target genes, common variants, and sequences. The sequences include RNA wings flanking DNA nucleotide sequences with 2′-O-Methylated RNA bases and phosphorothioate bonds. These sequences target variants of the MYH7 human gene.

FIG. 17 provides an example sequence listings for RNAi therapeutics (i.e., ASOs) implemented and experimented with according to several embodiments of the invention. These listings provide for means of targeting common variants of deleterious alleles and offer a promise of improved treatment of genetic disorders. As shown, sequence listing 1700 includes a list of sequence IDs, target genes, common variants, and sequences. The sequences include RNA wings flanking DNA nucleotide sequences with 2′-O-Methylated RNA bases and phosphorothioate bonds. These sequences target variants of the MYBPC3 human gene.

FIG. 18 provides an example sequence listings for RNAi therapeutics (i.e., ASOs) implemented and experimented with according to several embodiments of the invention. These listings provide for means of targeting common variants of deleterious alleles and offer a promise of improved treatment of genetic disorders. As shown, sequence listing 1800 includes a list of sequence IDs, target genes, common variants, and sequences. The sequences include RNA wings flanking DNA nucleotide sequences with 2′-O-Methylated RNA bases and phosphorothioate bonds. These sequences target variants of the MYL2 human gene.

FIG. 19 provides an example sequence listings for RNAi therapeutics (i.e., ASOs) implemented and experimented with according to several embodiments of the invention. These listings provide for means of targeting common variants of deleterious alleles and offer a promise of improved treatment of genetic disorders. As shown, sequence listing 1900 includes a list of sequence IDs, target genes, common variants, and sequences. The sequences include RNA wings flanking DNA nucleotide sequences with 2′-O-Methylated RNA bases and phosphorothioate bonds. These sequences target variants of the TNNI3 human gene.

FIG. 20 provides an example sequence listings for RNAi therapeutics (i.e., ASOs) implemented and experimented with according to several embodiments of the invention. These listings provide for means of targeting common variants of deleterious alleles and offer a promise of improved treatment of genetic disorders. As shown, sequence listing 2000 includes a list of sequence IDs, target genes, common variants, and sequences. The sequences include RNA wings flanking DNA nucleotide sequences. These sequences target variants of the TPM1 human gene.

FIG. 21 provides an example sequence listings for RNAi therapeutics (i.e., ASOs) implemented and experimented with according to several embodiments of the invention. These listings provide for means of targeting common variants of deleterious alleles and offer a promise of improved treatment of genetic disorders. As shown, sequence listing 2100 includes a list of sequence IDs, target genes, common variants, and sequences. The sequences include RNA wings flanking DNA nucleotide sequences. These sequences target variants of the TNNT2 human gene.

### Servers and Computer Systems

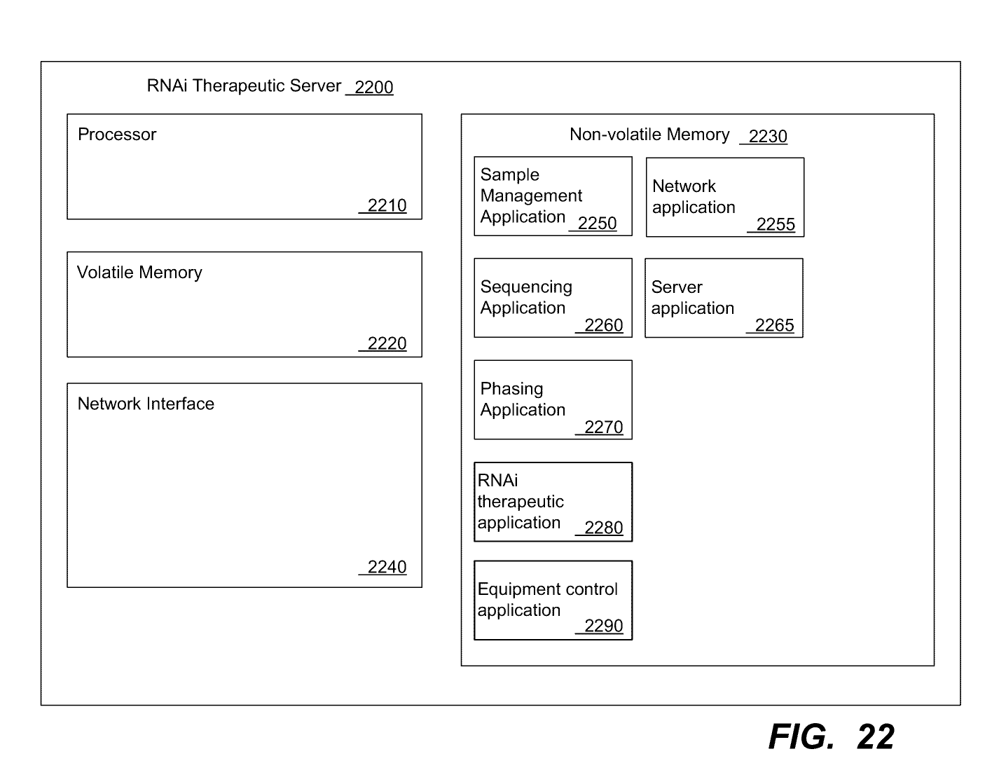

FIG. 22 is a hardware diagram of an RNAi therapeutic server in accordance with embodiments of the invention. An architecture of an RNAi therapeutic server in accordance with an embodiment of the invention is illustrated in FIG. 22. The RNAi therapeutic server manages the discovery, generation, and distribution of RNAi therapeutics in accordance with the various embodiments of the invention described above. The RNAi therapeutic server 2200 includes a processor 2210 in communication with non-volatile memory 2230, volatile memory 2220, and a network interface 2240. In the illustrated embodiment, the non-volatile memory includes a sample management application 2250, a network application 2255, a sequencing application 2260, a server application 2265, a phasing application 2270, an RNAi therapeutic application 2280, and an equipment control application 2290. The sample management application 2250 can perform operations including (but not limited to) sample intake handling, sample parsing, sample containerizing, and/or sorting of samples. The samples can be subject and/or patient samples. The network application 2255 can perform operations including (but not limited to) communication with other servers, systems, databases, cloud applications, virtual networks, networks, and/or the internet through the network interface 2240. The sequencing application 2260 can perform operations including (but not limited to) the sequencing operations discussed above in connection with process 200, process 300, workflow 700, workflow 800, and/or any other sequencing operations. The sequencing application 2260 can also interact with sequencing programs provided by third parties. The server application 2265 can perform operations including (but not limited to) run-time, support, and/or operating systems functionality necessary to run the RNAi therapeutic server 2200. The phasing application 2270 can perform operations including (but not limited to) phasing operations discussed above in connection with process 200, process 300, workflow 700, workflow 800, and/or any other phasing operations. The sequencing application 2260 can also interact with phasing programs provided by third parties. The RNAi therapeutic application 2280 can manage any operations in accordance with various embodiments of the invention as necessary to facilitate the remainder of the applications within RNAi therapeutic server 2200, including (but not limited to) operations discussed above in connection with process 200, process 300, workflow 700, workflow 800, and/or any other phasing operations. The equipment control application 2290 can perform operations including (but not limited to) management of physical lab devices utilized by an RNA therapeutic system.

In several embodiments, the network interface 2240 may be in communication with the processor 2210, the volatile memory 2220, and/or the non-volatile memory 2230. Although a specific RNAi therapeutic server architecture is illustrated in FIG. 22, any of a variety of architectures including architectures where the relation process is located on disk or some other form of storage and is loaded into volatile memory at runtime can be utilized to implement RNAi therapeutic servers in accordance with embodiments of the invention.

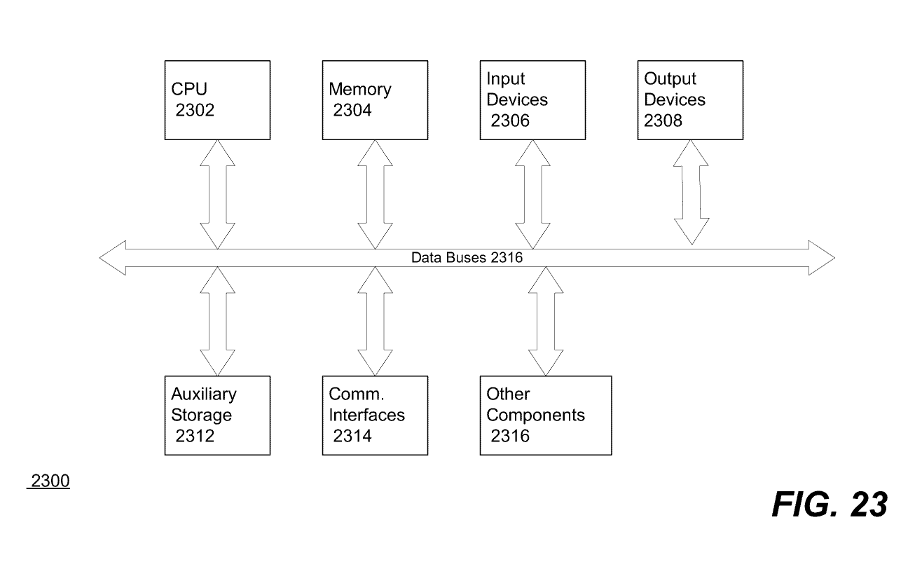

FIG. 23 is a computer system diagram in accordance with embodiments of the invention. Such a computer system is well-known in the art and may include the following. Computer system 2300 may include at least one central processing unit 2302 but may include many processors or processing cores. Computer system 2300 may further include memory 2304 in different forms such as RAM, ROM, hard disk, optical drives, and removable drives that may further include drive controllers and other hardware. Auxiliary storage 2312 may also be include that can be similar to memory 2304 but may be more remotely incorporated such as in a distributed computer system with distributed memory capabilities.

Computer system 2300 may further include at least one output device 2308 such as a display unit, video hardware, or other peripherals (e.g., printer). At least one input device 2306 may also be included in computer system 2300 that may include a pointing device (e.g., mouse), a text input device (e.g., keyboard), or touch screen.

Communications interfaces 2314 also form an important aspect of computer system 2300 especially where computer system 2300 is deployed as a distributed computer system. Computer interfaces 2314 may include LAN network adapters, WAN network adapters, wireless interfaces, Bluetooth interfaces, modems and other networking interfaces as currently available and as may be developed in the future.

Computer system 2300 may further include other components 2316 that may be generally available components as well as specially developed components for implementation of the present invention. Importantly, computer system 2300 incorporates various data buses 2316 that are intended to allow for communication of the various components of computer system 2300. Data buses 2316 include, for example, input/output buses and bus controllers.

Indeed, the present invention is not limited to computer system 2300 as known at the time of the invention. Instead, the present invention is intended to be deployed in future computer systems with more advanced technology that can make use of all aspects of the present invention. It is expected that computer technology will continue to advance but one of ordinary skill in the art will be able to take the present disclosure and implement the described teachings on the more advanced computers or other digital devices such as mobile telephones or “smart” televisions as they become available. Moreover, the present invention may be implemented on one or more distributed computers. Still further, the present invention may be implemented in various types of software languages including C, C++, and others. Also, one of ordinary skill in the art is familiar with compiling software source code into executable software that may be stored in various forms and in various media (e.g., magnetic, optical, solid state, etc.). One of ordinary skill in the art is familiar with the use of computers and software languages and, with an understanding of the present disclosure, will be able to implement the present teachings for use on a wide variety of computers.

The present disclosure provides a detailed explanation of the present invention with detailed explanations that allow one of ordinary skill in the art to implement the present invention into a computerized method. Certain of these and other details are not included in the present disclosure so as not to detract from the teachings presented herein but it is understood that one of ordinary skill in the art would be familiar with such details.

### DOCTRINE OF EQUIVALENTS

Those skilled in the art will appreciate that the foregoing examples and descriptions of various embodiments of the present invention are merely illustrative of the invention as a whole, and that variations in the steps and various components of the present invention may be made within the spirit and scope of the invention. Accordingly, the present invention is not limited to the specific embodiments described herein but, rather, is defined by the scope of the appended claims. Moreover, where processes, workflows, and/or techniques are described as being capable of being performed in accordance with embodiments of the invention, said embodiments may be freely combined, reordered, and/or substituted with each other without departing from the spirit and scope of the invention. In addition, while specific reference is made to HCM, the MYH7 gene, and the R403Q mutation of the MYH7 gene; many embodiments of the invention are readily applicable to different diseases, different genes, and/or different mutations.

1. A method of downregulating expression of deleteriously mutated alleles using RNAi therapeutics targeting common variants of alleles using an RNAi therapeutics system, the method comprising:
receiving subject samples using an RNAi therapeutics system, wherein the subject samples comprise a set of alleles from the subject;
sequencing the subject samples using the RNAi therapeutics system;
phasing the sequenced samples using the RNAi therapeutics system;
identifying a deleterious mutation on a particular allele from the set of alleles in the phased samples using the RNAi therapeutics system;
identifying a common variant in phase with the deleterious mutation on the particular allele using the RNAi therapeutics system;
selecting an RNAi therapeutic targeting the common variant using the RNAi therapeutics system; and
applying the selected RNAi therapeutic utilizing a vector and the RNAi therapeutics system.

2. The method of claim 1 further comprising:
selecting a disease using the RNAi therapeutics system;
identifying a gene upon which mutations can cause the selected disease using the RNAi therapeutics system;
identifying common variants of the gene using the RNAi therapeutics system; and
preparing RNAi therapeutics that target the identified common variants using the RNAi therapeutics system.

3. The method of claim 1, wherein sequencing the subject samples further comprising performing long read sequencing of the subject samples.

4. The method of claim 1, wherein the selected RNAi therapeutic comprises a therapeutic composition comprising:
a suitable vector; and
a nucleic acid compound with an antisense sequence complementary to RNA, wherein the nucleic acid compound includes 2′-O-Methylated RNA bases and phosphorothioate Bonds, and wherein the antisense sequence is any one of Seq. ID Nos. 1-27.

5. The method of claim 4, wherein the antisense sequence complementary to RNA is an antisense oligonucleotide targeting common variants of the Myosin Heavy Chain 7 (MYH7) gene.

6. The method of claim 1, wherein the received subject samples are from a patient with hypertrophic cardiomyopathy that has a deleteriously mutated gene.

7. The method of claim 6, wherein the deleteriously mutated gene is Myosin Heavy Chain 7 (MYH7) and the mutation is R403Q.

8. The method of claim 1, wherein the identified common variant is one of the following:
rs2069540 reference genotype,
rs2069540 alternate genotype,
rs58290801 reference genotype,
rs58290801 alternate genotype,
rs2239577 reference genotype,
rs2239577 alternate genotype,
rs2231124 reference genotype,
rs2231124 alternate genotype,
rs7145023 reference genotype,
rs7145023 alternate genotype,
rs3729823 reference genotype,
rs3729823 alternate genotype,
rs201797477 reference genotype,
rs201797477 alternate genotype,
rs2754155 reference genotype,
rs2754155 alternate genotype,
rs1951154 reference genotype,
rs1951154 alternate genotype,
rs7157716 reference genotype,
rs7157716 alternate genotype,
rs3729830 reference genotype,
rs3729830 alternate genotype,
rs2231126 reference genotype,
rs2231126 alternate genotype,
rs2231126 reference genotype,
rs144420313 alternate genotype, and
rs144420313 reference genotype.

9. The method of claim 1, wherein the vector comprises a transfection reagent.

10. An RNAi therapeutic system comprising:
at least one processing unit;
a memory storing a RNAi therapeutic application;
wherein the RNAi therapeutic application directs the at least one processing unit to:
receive subject samples, wherein the subject samples comprise a set of alleles from the subject;
sequence the subject samples;
phase the sequenced samples;
identify a deleterious mutation on a particular allele from the set of alleles in the phased samples;
identify a common variant in phase with the deleterious mutation on the particular allele;
select an RNAi therapeutic targeting the common variant; and
apply the selected RNAi therapeutic utilizing a vector.

11. (canceled)

12. The RNAi therapeutic system of claim 10, wherein the RNAi therapeutic application further directs the at least one processing unit to:
select a disease;
identify a gene upon which mutations can cause the selected disease;
identify common variants of the gene; and
prepare RNAi therapeutics that target the identified common variants.

13. The RNAi therapeutic system of claim 10, wherein sequencing the subject samples further comprising performing long read sequencing of the subject samples.

14. The RNAi therapeutic system of claim 10, wherein the selected RNAi therapeutic comprises a therapeutic composition comprising:
a suitable vector; and
a nucleic acid compound with an antisense sequence complementary to RNA, wherein the nucleic acid compound includes 2′-O-Methylated RNA bases and phosphorothioate Bonds, and wherein the antisense sequence is any one of Seq. ID Nos. 1-27.

15. The RNAi therapeutic system of claim 14, wherein the antisense sequence complementary to RNA is an antisense oligonucleotide targeting common variants of the Myosin Heavy Chain 7 (MYH7) gene.

16. The RNAi therapeutic system of claim 10, wherein the received subject samples are from a patient with hypertrophic cardiomyopathy that has a deleteriously mutated gene.

17. The RNAi therapeutic system of claim 16, wherein the deleteriously mutated gene is Myosin Heavy Chain 7 (MYH7) and the mutation is R403Q.

18. The RNAi therapeutic system of claim 10, wherein the identified common variant is one of the following:
rs2069540 reference genotype,
rs2069540 alternate genotype,
rs58290801 reference genotype,
rs58290801 alternate genotype,
rs2239577 reference genotype,
rs2239577 alternate genotype,
rs2231124 reference genotype,
rs2231124 alternate genotype,
rs7145023 reference genotype,
rs7145023 alternate genotype,
rs3729823 reference genotype,
rs3729823 alternate genotype,
rs201797477 reference genotype,
rs201797477 alternate genotype,
rs2754155 reference genotype,
rs2754155 alternate genotype,
rs1951154 reference genotype,
rs1951154 alternate genotype,
rs7157716 reference genotype,
rs7157716 alternate genotype,
rs3729830 reference genotype,
rs3729830 alternate genotype,
rs2231126 reference genotype,
rs2231126 alternate genotype,
rs2231126 reference genotype,
rs144420313 alternate genotype, and
rs144420313 reference genotype.

19. The RNAi therapeutic system of claim 10, wherein the vector comprises a transfection reagent.
